(19) United States
(12) Patent Application Publication
(10) Pub. No.: US 2026/0224733 A1
(43) Pub. Date: Aug. 6, 2026

(54) NANOPARTICLES DIRECTED TO CXCR4 AND USE THEREOF

(71) Applicant: TECHNION RESEARCH & DEVELOPMENT FOUNDATION LIMITED, Haifa (IL)

(72) Inventors: Yuval Yechiel SHAKED, Haifa (IL); Avi SCHROEDER, Haifa (IL); Rotem MENACHEM, Haifa (IL)

(21) Appl. No.: 19/151,428

(22) PCT Filed: Jan. 30, 2024
(86) PCT No.: PCT/IL2024/050116

§ 371 Date: Jul. 28, 2025

Related U.S. Application Data
(60) Provisional application No. 63/441,866, filed on Jan. 30, 2023.

(51) Int. Cl. A61K 47/69; A61K 9/1271; A61K 31/69; A61P 35/02

(52) U.S. Cl. CPC A61K 47/6911; A61K 9/1271; A61K 31/69; A61P 35/02

(57) ABSTRACT

Nanoparticles comprising an outer surface covalently conjugated to 1,4-Bis[(1,4,8,11-tetraazacyclotetradecan-1-yl)methyl]benzene (AMD3100) or a derivative thereof capable of binding to C-X-C chemokine receptor type 4 (CXCR4) are provided. Methods for of treating a CXCR4 positive cancer, such as multiple myeloma or acute myeloid leukemia, in a subject in need thereof, methods of determining suitability for treatment and methods of covalently linking a molecule comprising a secondary amine to a lipid nanoparticle, are also provided.

### CROSS REFERENCE TO RELATED APPLICATIONS

This application claims the benefit of priority of U.S. Provisional Patent Application No. 63/441,866 filed on Jan. 30, 2023, the contents of which are incorporated herein by reference in their entirety.

### FIELD OF INVENTION

The present invention is in the field of nanoparticles therapeutics.

### BACKGROUND OF THE INVENTION

Multiple myeloma (MM) is characterized by a neoplastic proliferation of plasma cells that is manifested primarily in the bone marrow. It accounts for almost 2% of cancer diagnoses and is the second most common hematological malignancy worldwide. MM is considered as a non-curable disease with median overall survival of 5 years. One of the most useful drugs to treat MM is bortezomib, a proteasome inhibitor, combined sometimes with thalidomide, an immunomodulatory agent. Bortezomib has led to major improvements in the survival of patients, yet, some MM patients fail to respond to the therapy and develop de novo resistance. Even with the use of second-generation drugs of bortezomib or other combinatorial treatment including chemotherapy and targeted drugs as a second-line treatment, patients eventually develop acquired resistance and succumb to this disease.

Previous studies on bortezomib for the treatment of MM demonstrated potential mechanisms to explain drug resistance. Specifically, it was shown that in addition to MM intrinsic resistance to bortezomib, the host generates pro-tumorigenic effects in response to bortezomib. These effects contribute to MM aggressiveness and support MM expansion and growth. It was demonstrated that the MM microenvironment in the bone marrow compartment is enriched with pro-inflammatory macrophages, which secrete factors such as IL-16 and IL-16 contributing to MM expansion and their stemness, respectively, further explaining the development of bortezomib resistance. These studies indicate that the bone marrow microenvironment in MM supports MM aggressiveness, especially after therapy, and enhances the ability of the cells to become resistant to therapy. Overall, the intrinsic and extrinsic mechanisms of MM resistance to bortezomib therapy require the development of new approaches and therapeutic strategies to overcome bortezomib resistance.

Over the years, anti-cancer drugs suffer from various limitations including low targeting ability, poor solubility, poor bioavailability, systemic toxicity, and acquired resistance. Lipid nanoparticles (LNPs) such as liposomes containing anti-cancer drugs have been developed to overcome these critical limitations. The unique properties of LNPs and their high surface-area-to-volume ratio allow the encapsulation of small molecule inhibitors, such as bortezomib. Different LNPs formulations can improve stability, permeability and drug release, reduce body clearance, and minimize off-target toxicity. In addition, coating LNPs surface with polyethylene glycol (PEG) extends their blood-circulation time and induce also immune tolerance. In solid tumors, a limited accumulation of nanocarriers is achieved by a passive uptake via the enhanced permeability and retention (EPR) effect. However, relying solely on EPR effect does not capitalize the full potential of liposomal-based drugs, therefore efforts are carried out for the use of targeting molecules that are conjugated to the surface of the nanocarriers to increase their affinity to tumor cells, and thus to facilitate a more specific drug uptake by cancer cells.

C-X-C chemokine receptor 4 (CXCR4) is a chemokine receptor for stromal cell-derived factor 1 (SDF-1). CXCR4 is expressed by different immune cells and has a role in facilitating hematopoietic stem cells (HSC) anchoring to the bone marrow. Moreover, CXCR4 is frequently over-expressed in various types of cancer and is known to be significantly associated with poorer progression-free survival (PFS) or overall survival (OS) in subjects with cancer, mainly due to its key role in metastasis development.

1,4-Bis[(1,4,8,11-tetraazacyclotetradecan-1-yl)methyl] benzene also known as AMD3100 or Plerixafor and sold under the brand name Mozobil®, a CXCR4 antagonist, an approved FDA drug, is currently being used for the mobilization of HSCs before bone marrow transplantation. Most MM cells overexpress CXCR4, and therefore CXCR4 can be used as a potential candidate for MM targeting purposes, especially in the bone marrow microenvironment. A previous study demonstrated the enhanced activity of the combination of AMD3100 with bortezomib, both administered as free drugs, for the treatment of MM. While the study demonstrated better efficacy than using bortezomib alone, the incidence of resistance in preclinical models has not changed, therefore raising the concern that AMD3100 acts in a different mechanism on MM cells to increase its therapeutic activity. In this regard, the effect of AMD3100 as a targeting molecule for bortezomib therapy using LNPs, and not as a free drug, has never been developed and its therapeutic activity has never been demonstrated.

There is an urgent need for new therapies for treating CXCR4 positive cancers, such as MM. Specifically, there is an unmet need to develop proteasome inhibitors-based drugs, such as bortezomib, that are target-directed, and that can overcome drug resistance and cancer recurrence due to low pharmacokinetics, off-target adverse effects, as well as pro-tumorigenic effects mediated by the proteasome inhibitor, when administrated in its free form. Thus, new treatment modalities which enhance the activity of bortezomib are required.

### SUMMARY OF THE INVENTION

The present invention provides nanoparticles comprising an outer surface covalently conjugated to 1,4-Bis[(1,4,8,11-tetraazacyclotetradecan-1-yl)methyl]benzene (AMD3100) or a derivative thereof capable of binding to C-X-C chemokine receptor type 4 (CXCR4). Methods for treating a CXCR4 positive cancer in a subject in need thereof, such as multiple myeloma or acute myeloid leukemia, methods of determining suitability for treatment and methods of covalently linking a molecule comprising a secondary amine to a lipid nanoparticle, are also provided.

A nanoparticle targeted to the bone marrow, and capable of delivering medications locally—such as small molecule drugs, proteins and nucleic acids.

According to a first aspect, there is provided a nanoparticle comprising an outer surface covalently conjugated to 1,4-Bis[(1,4,8,11-tetraazacylotetradecan-1-yl)methyl]benzene (AMD3100) or a derivative thereof capable of binding to C-X-C chemokine receptor type 4 (CXCR4).

According to some embodiments, the nanoparticle is a lipid nanoparticle.

According to some embodiments, the lipid nanoparticle is a liposome.

According to some embodiments, the lipid nanoparticle comprises a lipid, cholesterol, and polyethylene glycol (PEG)-lipid.

According to some embodiments, the lipid nanoparticle comprises hydrogenated soybean phosphatidylcholine (HSPC), cholesterol, and 1,2-Distearoyl-sn-glycero-3-phosphorylethanolamine (DSPE) covalently bound to PEG.

According to some embodiments, the nanoparticle comprises DSPE-PEG1000 and wherein the HSPC, cholesterol, DSPE-PEG 1000 are present at a molar ratio of 55:40:5, respectively.

According to some embodiments, the nanoparticle comprises PEG and the covalently conjugated is via an amide bond to a carboxyl group on the PEG.

According to some embodiments, conjugation is via EDC carbodiimide crosslinking with S-NHS between a carboxyl group on the PEG and an amide in the AMD3100.

According to some embodiments, the nanoparticle further comprises a drug.

According to some embodiments, the nanoparticle comprises an aqueous interior comprising the drug.

According to some embodiments, the drug is functional within a cell.

According to some embodiments, the drug is an anticancer drug.

According to some embodiments, the anticancer drug is a proteasome inhibitor.

According to some embodiments, the proteasome inhibitor is bortezomib.

According to some embodiments, the aqueous interior comprises a basic pH, optionally wherein the basic pH is about 9.5.

According to some embodiments, the nanoparticle comprises a bortezomib concentration of about 1 mg/ml.

According to some embodiments, the nanoparticle is for use in treating a CXCR4 positive cancer.

According to some embodiments, the CXCR4 positive cancer is selected from multiple myeloma and acute myeloid leukemia.

According to some embodiments, the CXCR4 positive cancer is multiple myeloma.

According to another aspect, there is provided a pharmaceutical composition comprising a nanoparticle of the invention and a pharmaceutically acceptable carrier, excipient or adjuvant.

According to another aspect, there is provided a method of treating a CXCR4 positive cancer in a subject in need thereof, the method comprising, administering to the subject a nanoparticle of the invention or a pharmaceutical composition of the invention, thereby treating a CXCR4 positive cancer.

According to some embodiments, the CXCR4 positive cancer is selected from multiple myeloma and acute myeloid leukemia.

According to some embodiments, the CXCR4 positive cancer is multiple myeloma.

According to some embodiments, the method comprises administering a therapeutically effective dose of the nanoparticle.

According to some embodiments, the nanoparticle comprises bortezomib and the dose is equivalent to a mouse dose of between 1-3 mg/kg body weight.

According to some embodiments, the method further comprises confirming surface expression of CXCR4 in cells of the cancer.

According to some embodiments, the confirming comprises receiving a sample from the subject comprising cancer cells and measuring expression of CXCR4 on a surface of the cancer cells, wherein expression of CXCR4 above a predetermined threshold indicates the subject is suitable for the administering.

According to another aspect, there is provided a method of determining suitability of a subject to be treated by a method of the invention, the method comprising receiving a sample from the subject comprising cancer cells and measuring expression of CXCR4 on a surface of the cancer cells, wherein expression of CXCR4 above a predetermined threshold indicates the subject is suitable to be treated by a method of the invention.

A method of covalently linking a molecule comprising a secondary amine to a lipid nanoparticle, the method comprising:
a. providing a lipid nanoparticle comprising at least one carboxyl group;
b. reacting the provided lipid nanoparticle with sulfo-N-hydroxysulfosuccinimide (S-NHS) and a carbodiimide coupling agent to obtain S-NHS active ester; and
c. reacting the S-NHS active ester with a molecule comprising a secondary amine;
thereby linking conjugating the secondary amine to the lipid nanoparticle.

According to some embodiments, the covalently linking comprises forming an amide bond between the secondary amine and the carboxy of the lipid nanoparticle.

According to some embodiments, the lipid nanoparticle comprises PEG comprising the at least one carboxyl group, and the method comprises producing an amide bond between the PEG and the molecule.

According to some embodiments, the secondary amine is a tertiary amide upon linking.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1F: Preparation and characterization of ATBL. (1A) Illustration of AMD Conjugation reaction was performed using EDC and NHS reaction. (1B) Comparison between passive and active loading of bortezomib liposome demonstrated in the graph. (1C) Illustration of bortezomib active loading into the liposomes by pH and concentration gradient is demonstrated. (1D) The size of liposomes was measured by dynamic light scattering (DLS). (1E) Encapsulation efficiency (% EE) of increasing bortezomib concentration for active loading is determined. (1F) Drug release of bortezomib over 10 days period is shown in the graph.

FIGS. 2A-2F: Therapeutic activity of ATBL is dependent on CXCR4. (2A) CAG cell migration in response to SDF-1 chemokine in the presence of AMD liposomes or AMD was assessed using the Boyden chamber migration assay. (2B-2C) An increased uptake of AMD liposomes in MM RPMI2886 and CAG cells assessed by Cy5 fluorescence was carried out by flow cytometry. Uptake of 1% (2B), and 3% (2C) liposomes are shown. (2D-2E) The late apoptosis profile of RPMI8226 (2D) and CAG (2E) cell lines was assessed by flow cytometry 48 h after treatment with ATBL. (2F) IC50 measurement of bortezomib as free drug (9.13 nM) and ATBL (5.96 nM).

FIG. 3: ATBL superior activity than the combination of AMD and bortezomib. Mice were treated with ATBL, the combination of free bortezomib and free AMD and were compared with the control untreated group. Tumor growth was determined by bioluminescence imaging.

FIG. 4: Superiority of covalent linkage of AMD to a bortezomib liposome. Mice were treated with ATBL, the combination of bortezomib liposomes and free AMD and bortezomib liposomes with AMD electrostatically adhered to their surface and were compared with the control untreated group. Tumor growth was determined by bioluminescence imaging.

FIG. 5: Zeta potential of ATBL displays an effective AMD binding to bortezomib liposomes. Adherence of positively charged AMD to liposomes effectively increased the zeta potential of the composition, however, after dialysis the AMD produced increase was lost when the AMD was adsorbed by electrostatic interaction but not when it was covalently linked to the liposome.

FIGS. 6A-6C: Biodistribution of liposomes with covalent AMD attachment to the bone marrow and multiple myeloma cells. (6A) Bar graph of Gd concentration in bone after IV administration to mice of untargeted liposomes or liposomes with covalent AMD conjugation loaded with Gd. (6B-6C) Bar graphs of the percent of (6B) bone marrow cells and (6C) multiple myeloma cells in the bone marrow positive for liposomes after administration of three different liposome preparations.

FIGS. 7A-7C: Biodistribution of AMD-liposomes to AML cells in the bone marrow. (7A) Bar graph of CXCR4 expression in multiple myeloma cells and AML cells. (7B-7C) Bar graphs of the percent of (7B) bone marrow cells and (7C) AML cells in the bone marrow positive for liposomes after administration of AMD-liposomes or non-targeted liposomes.

### DETAILED DESCRIPTION OF THE INVENTION

The present invention, in some embodiments, provides nanoparticles comprising an outer surface covalently conjugated to AMD3100 or a derivative thereof capable of binding to C-X-C chemokine receptor type 4 (CXCR4). Bortezomib containing nanoparticles with AMD3100 covalently conjugated to their surface are also provided. Methods for treating a CXCR4 positive cancer, such as multiple myeloma or acute myeloid leukemia, in a subject in need thereof are provided, as are methods of determining suitability of a subject for treatment and methods of covalently linking a molecule comprising a secondary amine to a lipid nanoparticle.

The invention is based, at least in part, on the surprising finding that molecules containing a secondary amide such as AMD3100 which are notoriously hard to conjugate to lipids can be successfully conjugated to the surface of a lipid nanoparticle. This conjugation was successfully achieved using S-NHS and a carbodiimide coupling agent when the lipid was modified with a carboxyl group. The successfully surface conjugation allowed for the production of CXCR4 targeted nanoparticles that when administered systemically homed to CXCR4 positive cancer. When AMD3100 is only adsorbed to the surface the molecule can be lost in the bloodstream and targeting is greatly reduced.

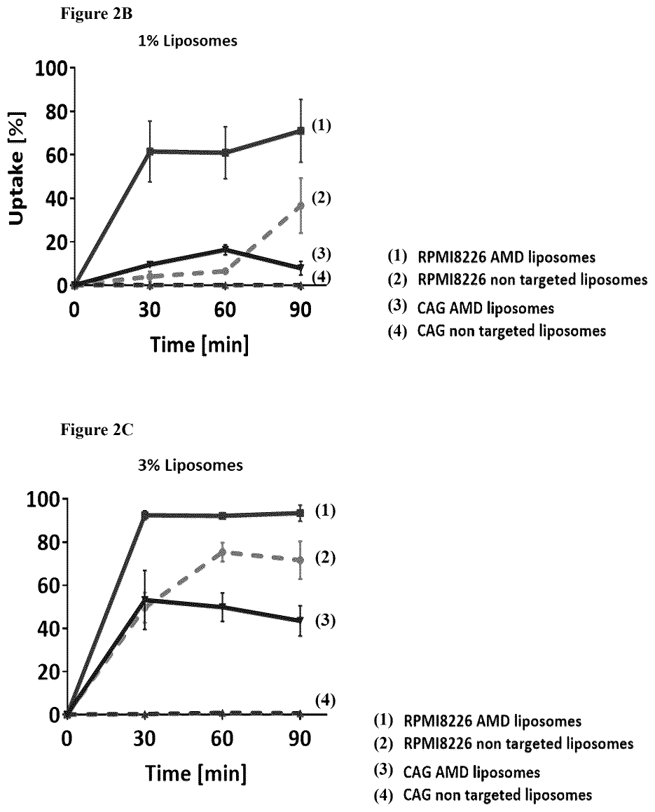

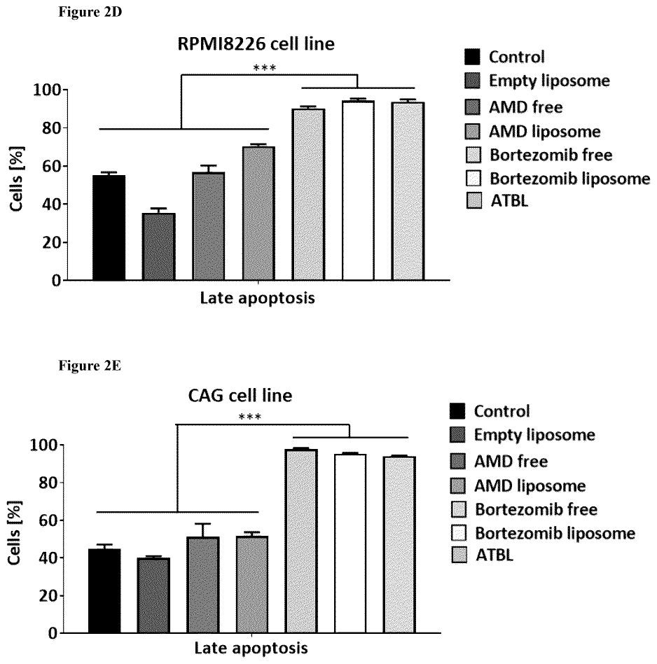

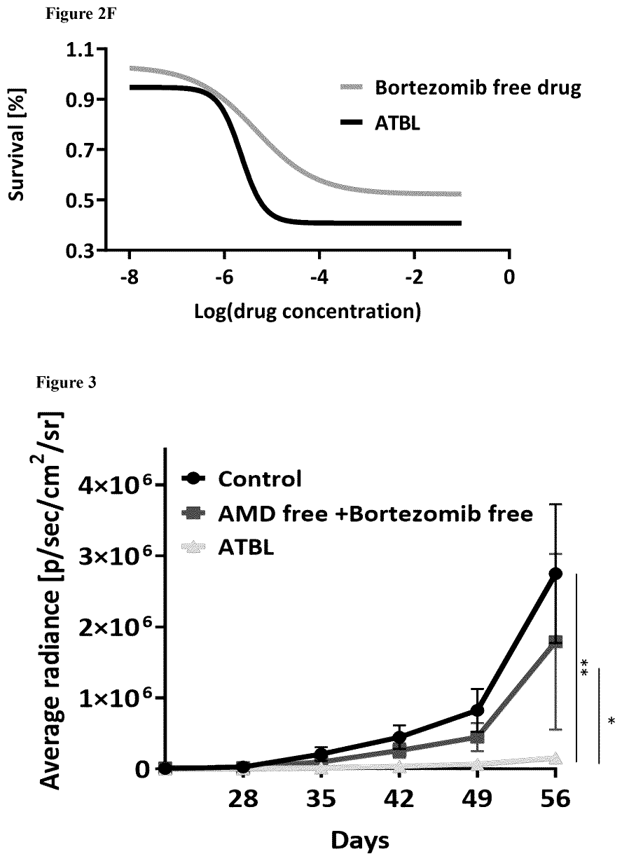

It was further surprisingly found that the conjugation of AMD3100 to the surface of the nanoparticle enhanced drug (e.g., bortezomib) uptake into the cell (FIG. 2B-2C). The surprising result was that in vivo treatment with an AMD3100 conjugated nanoparticle encapsulating bortezomib was statistically significantly superior to treatment with a combination of free AMD3100 and free bortezomib. Indeed, the AMD3100 conjugated, bortezomib containing nanoparticles nearly completely inhibited tumor growth (FIG. 3).

### Nanoparticles

By a first aspect, there is provided a nanoparticle comprising an outer surface conjugated to an antagonist of C-X-C chemokine receptor type 4 (CXCR4). In some embodiments, the nanoparticle disclosed herein comprises an outer surface covalently conjugated to the CXCR4 receptor antagonist. In some embodiments, the nanoparticle disclosed herein comprises an outer surface covalently conjugated to a protein that binds to CXCR4. In some embodiments, the nanoparticle disclosed herein comprises an outer surface covalently conjugated to a small molecule that binds to CXCR4.

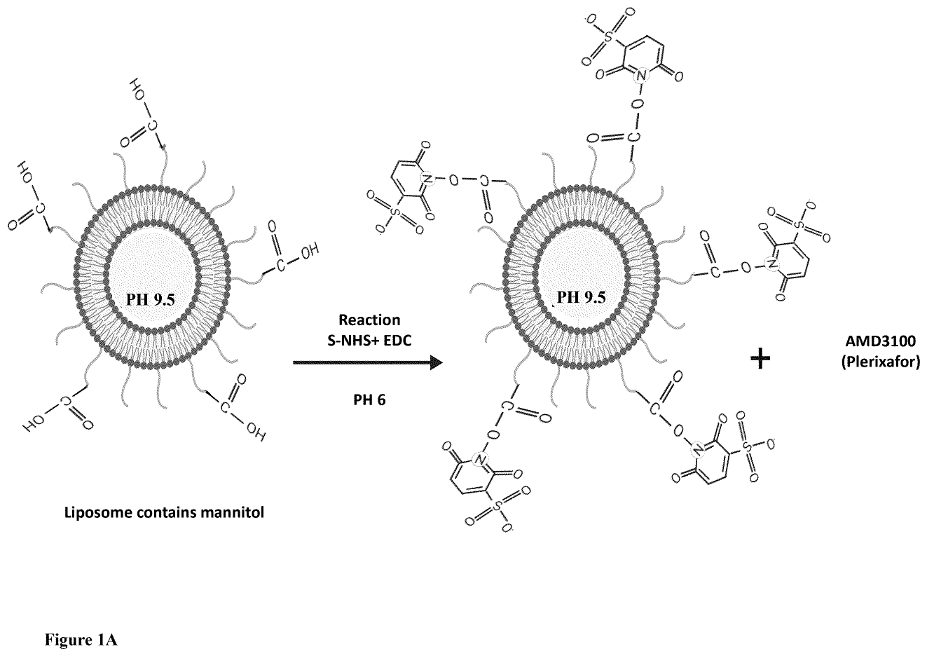

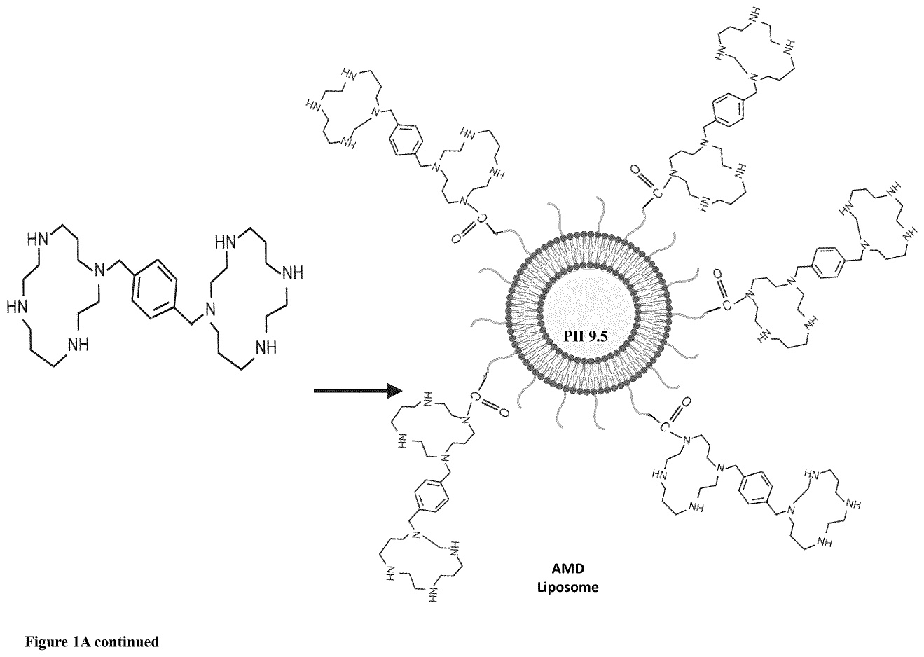

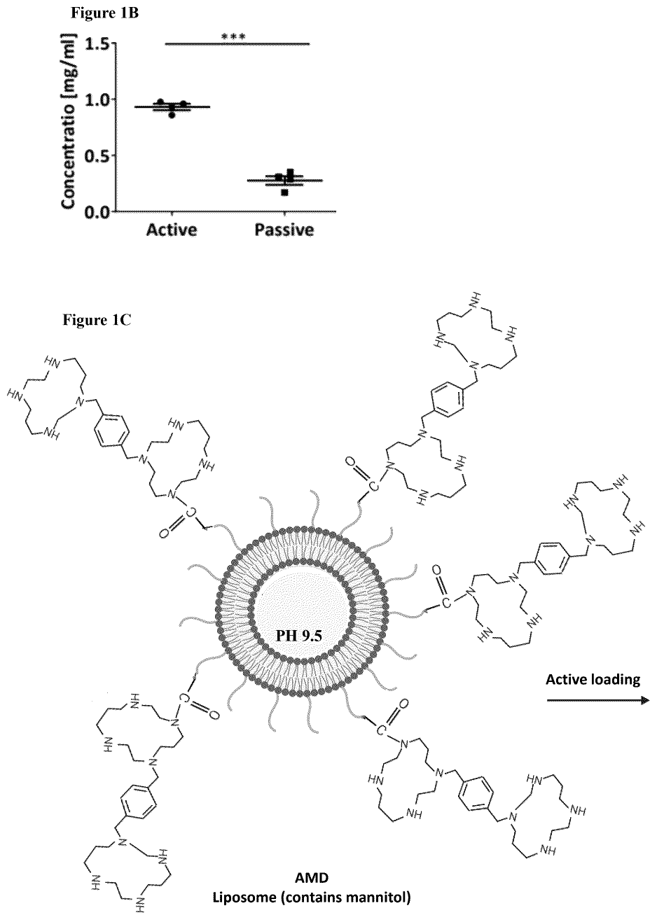

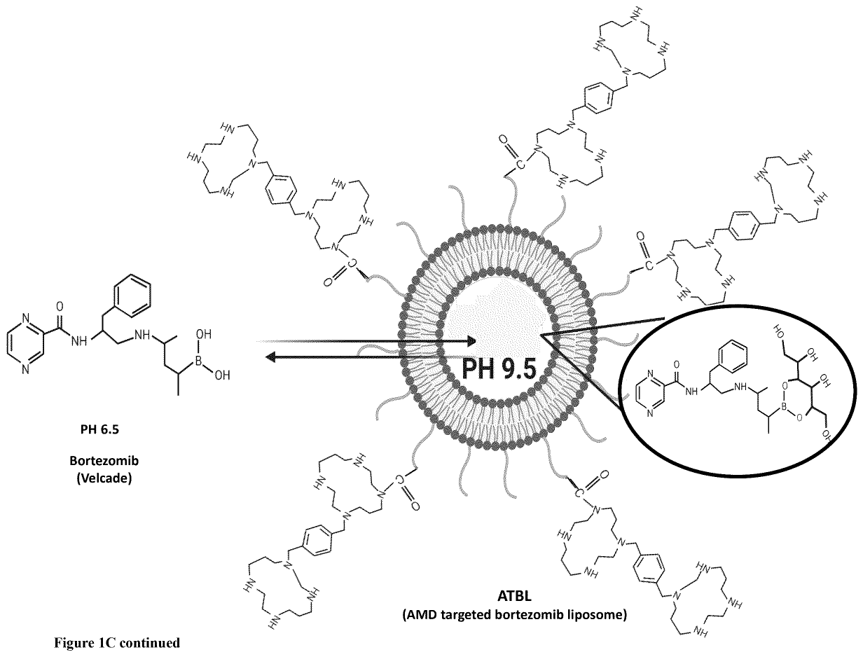

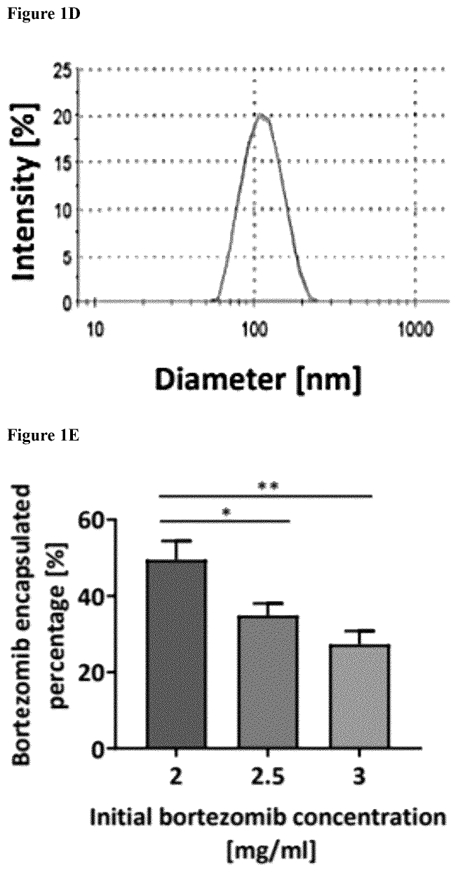

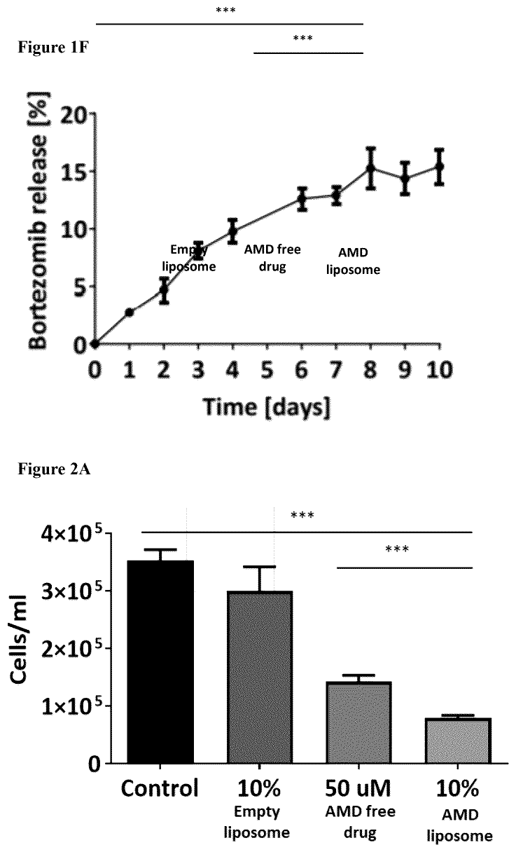

In some embodiments, the CXCR4 antagonist comprises 1,4-Bis[(1,4,8,11-tetraazacyclotetradecan-1-yl)methyl]benzene. 1,4-Bis[(1,4,8,11-tetraazacyclotetradecan-1-yl)methyl]benzene is also known as AMD3100 and these two names will be used interchangeably herein. The structure of this small molecule antagonist is provided in FIG. 1A and can be found under CAS 110078-46-1. The empirical formula for AMD3100 is C28H54N8. AMD3100 is also known as CXCR4 antagonist I-AMD3100, JM3100, Plerixafor, and SID791, and is sold under the trade name Mozobil®. AMD3100 is commercially available from retailers such as Abcam, Sigma Aldrich, Millipore, R&D Systems and many others. In some embodiments, the CXCR4 antagonist is AMD3100. In some embodiments, the nanoparticle comprises an outer surface conjugated to AMD3100. In some embodiments, the nanoparticle comprises an outer surface conjugated to a derivative of AMD3100. In some embodiments, conjugated is covalently conjugated.

As used here, the term “derivative” refers to small molecules based of the structure of AMD3100 and includes salts, hydrates, isomers, enantiomers, and polymorphs thereof. In some embodiments, the derivative is a salt of AMD3100. Salts of AMD3100 are well known in the art and include for example AMD3100 octahydrochloride hydrate (CAS: 155148-31-5). In some embodiments, an AMD3100 derivative is capable of binding to CXCR4. In some embodiments, an AMD3100 derivative is capable of binding to CXCR4 at at least the same level as AMD3100. In some embodiments, a derivative comprises the double ring structure of AMD3100. In some embodiments, a derivative comprises a secondary amide. In some embodiments, a derivative comprises a ring comprising 3 secondary amides. In some embodiments, the ring comprises 1 tertiary amide. In some embodiments, a derivative comprises two rings. In some embodiments, the rings of a derivative are separated by a six-carbon ring. In some embodiments, a derivative comprises AMD3100 and an additional side chain. In some embodiments, the derivative is not BAT1. BAT1 is disclosed in McCallion et al., “Dual-action CXCR4-targeting liposomes in leukemia: function blocking and drug delivery”, Blood Adv., 2019, Jul. 23; 3(14):2069-81, herein incorporated by reference in its entirety.

As used herein, the term “CXCR4 antagonist” encompasses a substance which selectively binds the CXCR4 receptor on a cell surface, and reduces, hinders, or prevents the cell's activation by CXCR4. In some embodiments, the CXCR4 antagonist prevents the receptor's ligand binding to CXCR4 receptor. In some embodiments, the CXCR4 antagonist binds to or occludes the ligand binding pocket of CXCR4. In some embodiments, a CXCR4 ligand is stromal derived factor 1 (SDF-1/CXCL12). In some embodiments, the CXCR4 receptor antagonist attenuates downstream effects of SDF-1 in the cell. In some embodiments, the downstream effect is activation of the cell. Examples for CXCR4 receptor antagonists are known in the art and any such inhibitor may be used. Non-limiting examples include: AMD3100, BL-8040 (motixafortide, CAS NO: 664334-36-5), and AZD2098(2,3-dichloro-N-(3-methoxypyrazin-2-yl)benzenesulfonamide, EC NO: 938-242-2.

In some embodiments, the nanoparticle disclosed herein is a lipid nanoparticle (LNP). As used herein, a “lipid nanoparticle” is a nanoparticle that comprises a lipid. In some embodiments, the shell of the nanoparticle comprises the lipid. In some embodiments, the outer surface comprises the lipid. In some embodiments, the outer surface is a lipid layer. In some embodiments, the lipid nanoparticle comprises at least one lipid layer. In some embodiments, the lipid nanoparticle comprises two lipid layers (e.g., a lipid bilayer). In some embodiments, the lipid nanoparticle comprises a lipid bilayer. In some embodiments, the lipid nanoparticle comprises at least one phospholipid layer. In some embodiments, the lipid nanoparticle comprises two phospholipid layers. In some embodiments, the lipid nanoparticle is spherical.

In some embodiments, the lipid nanoparticle comprises a diameter between 10 and 1000 nanometers (nm). In some embodiments, the lipid nanoparticle comprises a diameter between 50 and 500 nm, between 50 and 450 nm, between 50 and 400 nm, between 50 and 350 nm, between 50 and 300 nm, between 50 and 250 nm, between 50 and 200 nm, between 50 and 150 nm, between 60 and 500 nm, between 60 and 450 nm, between 60 and 400 nm, between 60 and 350 nm, between 60 and 300 nm, between 60 and 250 nm, between 60 and 200 nm, between 60 and 150 nm, between 70 and 500 nm, between 70 and 450 nm, between 70 and 400 nm, between 70 and 350 nm, between 70 and 300 nm, between 70 and 250 nm, between 70 and 200 nm, between 70 and 150 nm, between 50 and 140 nm, between 60 and 140 nm, between 70 and 140 nm, between 50 and 130 nm, between 60 and 130 nm, or between 70 and 130 nm. Each possibility represents a separate embodiment of the present invention. In some embodiments, the lipid nanoparticle comprises a diameter between 67 nm to 137 nm (107±30 nm).

In some embodiments, the lipid nanoparticle comprises an aqueous interior. In some embodiments, the interior is a core. In some embodiments, the aqueous interior is an aqueous solution. In some embodiments, the lipid nanoparticle comprises an aqueous core. In some embodiments, the aqueous core is surrounded by a hydrophobic membrane. In some embodiments, the hydrophobic membrane comprises at least one lipid layer. In some embodiments, the lipid layer comprises a phospholipid layer.

In some embodiments, the lipid nanoparticle comprises an ionizable lipid. Examples of ionizable lipids include, but are not limited to DLin-DMA, D-Lin-MC2-DMA, DLin-KC2-DMA, DLin-MC3-DMA, XTC, ALN100, MC3, C12-200, SM-102, ALC-0315, CL1, TCL053, CKK-E12, A9, and LP01. In some embodiments, the ionizable lipid binds to an uncharged cargo. In some embodiments, the uncharged cargo is an uncharged agent. In some embodiments, the uncharged cargo is an uncharged therapeutic. In some embodiments, the uncharged cargo is a nucleic acid molecule. In some embodiments, the ionizable lipid binds to a nucleic acid. In some embodiments, a nucleic acid is a nucleic acid molecule. In some embodiments, the nucleic acid molecule is selected from DNA and RNA. In some embodiments, the nucleic acid molecule is a synthetic nucleic acid molecule. In some embodiments, the nucleic acid molecule comprises a synthetic nucleic acid.

In some embodiments, the lipid nanoparticle comprises a liposome. In some embodiments, the lipid nanoparticle is a liposome. As used herein, the term “liposome” refers to an artificial vesicle, spherical in shape, having at least one lipid layer. In some embodiment, the liposome comprises a lipid bilayer. In some embodiments, the liposome comprises a phospholipid bilayer.

The terms “particle size” and “particle diameter” are used herein interchangeably and refer to an average cross section size of the nanoparticles (e.g., a largest linear distance between two points on the surface of the nanoparticle) within a liquid composition. In some embodiments, the term “average cross section size” may refer to either the average of at least e.g., 70%, 80%, 90%, or 95% of the particles, or in some embodiments, to the median size of the plurality of nanoparticles. In some embodiments, the term “average cross section size” refers to a number average of the plurality of nanoparticles. In some embodiments, the term “average cross section size” may refer to an average diameter of substantially spherical nanoparticles.

In some embodiments, the nanoparticle of the invention is or comprises a lipid-based particle. In some embodiments, the nanoparticle of the invention is or comprises a liposome. In some embodiments, liposomes refer to vesicles with an internal core surrounded by a lipid bilayer/s and are widely used as drug carriers. This is greatly due to their unique characteristics such as good biocompatibility, low toxicity, lack of immune system activation, and the ability to incorporate both hydrophobic and hydrophilic compounds. As described herein, liposomes are known in the art as artificial vesicles composed of a substantially spherical lipid bilayer which typically, but not exclusively, comprises phospholipids, sterol, e.g., cholesterol, and other lipids.

In some embodiments, the nanoparticle of the invention comprises a core and a shell encapsulating or enclosing the core. In some embodiments, the core is a hollow core, or a core filled with a solid or liquid material. In some embodiments, the nanoparticle of the invention may have a spherical or any other geometrical shape. In some embodiments, the nanoparticle of the invention comprises a unilamellar or multilamellar membrane (or lipid layer). In some embodiments, the liposomes disclosed herein can be any one or combination of vesicles selected from the group consisting of small unilamellar vesicles (SUV), large unilamellar vesicles (LUV), multilamellar vesicles (MLV), multivesicular vesicles (MVV), large multivesicular vesicles (LMVV, also referred to, at times, by the term giant multivesicular vesicles, “GMV”), oligolamellar vesicles (OLV), and others. In some embodiments, the liposomes are large unilameller vesicles (LUV). Methods of preparing and characterizing pharmaceutical liposome compositions are known in the field (see, e.g., Lasic D. Liposomes: From physics to applications, Elsevier, Amsterdam 1993; G. Greroriadis (ed.), Liposome Technology, 3rd edition, vol. 1-3, CRC Press, Boca Raton, 2006; Hong et al., U.S. Pat. No. 8,147,867, incorporated by reference herein in their entirety for all purposes).

In some embodiments, the liposomes are characterized by a proper packing parameter. As used herein and in the art, “packing parameter” is a relative measure of a given lipid composition, and depend on factors such as size relationships between lipid head groups and lipid hydrocarbon chains, charge, and the presence of stabilizers such as cholesterol. It should also be noted that the packing parameter may be not constant. In some embodiments, the parameter is dependent on various conditions which effect each the volume of the hydrophobic chain, the cross-sectional area of the hydrophilic head group, and the length of the hydrophobic chain. Factors can affect these include, but are not limited to, the properties of the solvent, the solvent temperature, and the ionic strength of the solvent. In some embodiments, the proper packing parameter is in the range of 0.3 to 1, e.g., 0.3, 0.5, 0.7, 0.9, or 1, including any value and range therebetween.

The term “core”, as used herein, refers to the central portion of the particle, with a different composition than the shell. In some embodiments, the core is enclosed by the shell. In some embodiments, the core is bound to the inner portion of the shell. In some embodiments, the core comprises a drug enclosed by the shell. In some embodiments, enclosed is encapsulated. In some embodiments, the core is a liquid. In some embodiments, the core comprises an aqueous solution. In some embodiments, the core comprises an aqueous solution of a compound as described herein. In some embodiments, the core comprises a compound as described herein, substantially located therewithin.

In some embodiments, the lipid nanoparticle comprises cholesterol. In some embodiments, the lipid nanoparticle comprises polyethylene glycol (PEG). In some embodiments, the lipid nanoparticle comprises a pegylated lipid. In some embodiments, the lipid nanoparticle comprises (PEG)-lipid. In some embodiments, the lipid nanoparticle comprises a lipid, cholesterol, and polyethylene glycol (PEG)-lipid.

In some embodiments, the lipid is or comprises one or more phospholipids. In some embodiments, the phospholipid is a liposome-forming lipid. As used herein, the term “liposome forming lipid” encompasses phospholipids which, upon dispersion or dissolution thereof in an aqueous solution at a temperature above a transition temperature (Tm), undergo self-assembly so as to form stable liposomes. As used herein, the term Tm refers to a temperature at which phospholipids undergo phase transition from solid (ordered phase, also termed as a gel phase) to a fluid (disordered phase, also termed as fluid crystalline phase). Tm also refers to a temperature (or to a temperature range) at which the maximal change in heat capacity occurs during the phase transition.

In some embodiments, the lipid nanoparticle comprises phosphatidylcholine. In some embodiments, the phosphatidylcholine comprises a soybean phosphatidylcholine. In some embodiments the phosphatidylcholine is hydrogenated. In some embodiments, the lipid nanoparticle comprises a hydrogenated soybean phosphatidylcholine (HSPC). In some embodiments, the phosphatidylcholine is HSPC. In some embodiments, the lipid nanoparticle comprises 1-palmitoyl-2-oleoyl-sn-glycero-3-phosphocholine (POPC). In some embodiments, the phosphatidylcholine is POPC. In some embodiments, the lipid nanoparticle comprises Dipalmitoylphosphatidylcholine (DPPC). In some embodiments, the phosphatidylcholine is DPPC. In some embodiments, the lipid nanoparticle comprises Distearoylphosphatidylcholine (DSPC). In some embodiments, the phosphatidylcholine is DSPC. In some embodiments, the lipid nanoparticle comprises dioleoylphosphatidylcholine (DOPC). In some embodiments, the phosphatidylcholine is DOPC. In some embodiments, the lipid nanoparticle comprises Dimyristoylphosphatidylcholine (DMPC). In some embodiments, the phosphatidylcholine is DMPC. In some embodiments, the lipid nanoparticle comprises a lipid selected from HSPC, POPC, DPPC, DSPC, DOPC and DMPC. It will be understood that for compositions comprising HSPC, the HSPC can be replaced with any of POPC, DPPC, DSPC, DOPC and DMPC. In some embodiments, the lipid nanoparticle comprises cholesterol. In some embodiments, the lipid nanoparticle comprises phosphoethanolamine (PE). In some embodiments, PE comprises 1,2-Distearoyl-sn-glycero-3-phosphorylethanolamine (DSPE). In some embodiments, the lipid nanoparticle comprises polyethylene glycol (PEG). In some embodiments, the lipid nanoparticle comprises polyethylene glycol (PEG) covalently bound to a lipid (PEG-lipid). In some embodiments, the lipid nanoparticle comprises polyethylene glycol (PEG) covalently bound to PE. In some embodiments, the lipid nanoparticle comprises polyethylene glycol (PEG) covalently bound to DSPE.

In some embodiments, the lipid nanoparticle comprises phosphatidylcholine, cholesterol, PEG and PE. In some embodiments, the lipid nanoparticle comprises phosphatidylcholine, cholesterol, and PEG-PE. In some embodiments, the lipid nanoparticle comprises HSPC, cholesterol, PEG and DSPE. In some embodiments, the lipid nanoparticle comprises HSPC, cholesterol, and PEG-DSPE. In some embodiments, PEG comprises PEG1000. In some embodiments, PEG is PEG2000, PEG1900, PEG1800, PEG1700, PEG1600, PEG1500, PEG1400, PEG1300, PEG1200, PEG1100, PEG1000, PEG900, PEG800, PEG700, PEG600, PEG500, PEG400, PEG300, PEG200 or PEG100. Each possibility represents a separate embodiment of the invention. In some embodiments, the PEG is a mix of PEGs of different lengths. In some embodiments, the PEG is a mix of PEG1000 and PEG2000. In some embodiments, the PEGs are present in equal percentages. In some embodiments, the lipid nanoparticle comprises HSPC, cholesterol, DSPE, and PEG1000. In some embodiments, the lipid nanoparticle comprises HSPC, cholesterol, and DSPE-PEG1000.

In some embodiments, the molar (m:m) ratio between HSPC to cholesterol in the nanoparticle disclosed herein is between 1:1 to 5:1. In some embodiments, the molar ratio between HSPC to cholesterol is between 1:1 to 4:1, between 1:1 to 3:1, or between 1:1 to 2:1. Each embodiment represents a separate embodiment of the present invention. In some embodiments the molar ratio between HSPC to cholesterol is between 1:1 to 1.9:1, between 1:1 to 1.8:1, between 1:1 to 1.7:1, between 1:1 to 1.6:1, between 1:1 to 1.5:1, between 1:1 to 1.4:1, between 1.1:1 to 1.9:1, between 1.1:1 to 1.8:1, between 1.1 to 1.7:1, between 1.1:1 to 1.6:1, between 1.1:1 to 1.5:1, between 1.1:1 to 1.4:1, between 1.2:1 to 1.9:1, between 1.2:1 to 1.8:1, between 1.2:1 to 1.7:1, between 1.2:1 to 1.6:1, between 1.2:1 to 1.5:1, between 1.2:1 to 1.4:1, between 1.3:1 to 1.9:1, between 1.3:1 to 1.8:1, between 1.3:1 to 1.7:1, between 1.3:1 to 1.6:1, between 1.3:1 to 1.5:1, or between 1.3:1 to 1.4:1. Each embodiment represents a separate embodiment of the present invention. In some embodiments the molar ratio between HSPC to cholesterol is about 1.375:1.

In some embodiments, the molar ratio of cholesterol to DSPE-PEG in the nanoparticle disclosed herein is between 4:1 to 12:1.In some embodiments, the molar ratio of cholesterol to DSPE-PEG is between 4:1 to 11:1, between 4:1 to 10:1, between 4:1 to 9:1, between 5:1 to 12:1, between 5:1 to 11:1, between 5:1 to 10:1, between 5:1 to 9:1, between 6:1 to 12:1, between 6:1 to 11:1, between 6:1 to 10:1, between 6:1 to 9:1, between 7:1 to 12:1, between 7:1 to 11:1, between 7:1 to 10:1, or between 7:1 to 9:1. Each embodiment represents a separate embodiment of the present invention. In some embodiments, the molar ratio of cholesterol to DSPE-PEG is about 8:1.

In some embodiments, the molar ratio of HSPC to DSPE-PEG in the nanoparticle disclosed herein is between 2:1 to 20:1. In some embodiments, the molar ratio of HSPC to DSPE-PEG in the nanoparticle disclosed herein is between 5:1 to 15:1, between 5:1 to 14:1, between 5:1 to 13:1, between 5:1 to 12:1, between 6:1 to 15:1, between 6:1 to 14:1, between 6:1 to 13:1, between 6:1 to 12:1, between 7:1 to 15:1, between 7:1 to 14:1, between 7:1 to 13:1, between 7:1 to 12:1, between 8:1 to 15:1, between 8:1 to 14:1, between 8:1 to 13:1, between 8:1 to 12:1, between 9:1 to 15:1, between 9:1 to 14:1, between 9:1 to 13:1, between 9:1 to 12:1, between 10:1 to 15:1, between 10:1 to 14:1, between 10:1 to 13:1, or between 10:1 to 12:1. Each embodiment represents a separate embodiment of the present invention. In some embodiments, the molar ratio of HSPC to DSPE-PEG is about 11:1.

In some embodiments, the molar ratio of HSPC, cholesterol, and DSPE-PEG in the nanoparticle disclosed herein is about 11:8:1, respectively. In some embodiments, the molar ratio of HSPC, cholesterol, and DSPE-PEG in the nanoparticle disclosed herein is about 55:40:5, respectively. In some embodiments, the PEG is half PEG1000 and half PEG2000. In some embodiments, the PEG is all PEG1000.

In some embodiment, the nanoparticle is a MC3-DLin-DMA:DSPC:Cholesterol:DMG-PEG2000 nanoparticle. In some embodiments, the ratio of MC3-DLin-DMA:DSPC:Cholesterol:DMG-PEG 2000 is 50:10:38.5:1.5. In some embodiments, the nanoparticle is a SM102:DSPC:Cholesterol:DMG-PEG2000 nanoparticle. In some embodiments, the ratio of SM102:DSPC:Cholesterol:DMG-PEG2000 is 50:10:38.5:1.5. In some embodiments, the nanoparticle is a ALC0315:DSPC:Cholesterol:ALC0159 nanoparticle. In some embodiments, the ratio of ALC 0315:DSPC:Cholesterol:ALC0159 is 50:10:38.5:1.5.

In some embodiments, the nanoparticle comprises PEG covalently conjugated to AMD3100. In some embodiments, the nanoparticle comprises PEG1000 covalently conjugated to AMD3100. In some embodiments, the covalent conjugation is via an amide in the AMD3100. In some embodiments, covalent conjugation is covalent linkage. some embodiments, the covalent linkage is via an amide in the AMD3100. In some embodiments, the covalent conjugation is via a carboxyl group on the PEG. In some embodiments, the covalent linkage is via a carboxyl group on the PEG. In some embodiments, the covalent conjugation is between an amide in the AMD3100 to a carboxyl group on the PEG. In some embodiments, the covalent linkage is between an amide in the AMD3100 to a carboxyl group on the PEG. In some embodiments, the covalent conjugation produces an amide bond. In some embodiments, the covalent linkage produces an amide bond. In some embodiments, the covalent conjugation is amide bonding. In some embodiments, the covalent conjugation is via an amide bond. In some embodiments, the covalent linkage is amide bonding. In some embodiments, the covalent linkage is via an amide bond. In some embodiments, covalent linkage is covalent binding.

In some embodiments, the conjugation is via carbodiimide crosslinking. In some embodiments, the linking is via carbodiimide crosslinking. In some embodiments, carbodiimide crosslinking is with a carbodiimide coupling agent. In some embodiments, the conjugation between a carboxyl group on the PEG and an amide in the AMD3100 is via carbodiimide crosslinking with a carbodiimide coupling agent. In some embodiments, the linking between a carboxyl group on the PEG and an amide in the AMD3100 is via carbodiimide crosslinking with a carbodiimide coupling agent. In some embodiments, the carbodiimide coupling agent comprises EDC. In some embodiments, the carbodiimide crosslinking is with sulfo-N-hydroxysulfosuccinimide (S-NHS). In some embodiments, the carbodiimide crosslinking is with NHS. As used herein, EDC (1-ethyl-3-(3-dimethylaminopropyl)carbodiimide hydrochloride), is a known carbodiimide coupling agent. Other non-limiting examples for carbodiimide coupling agents are dicyclohexylcarbodiimide (DCC) and diisopropylcarbodiimide (DIC).

In some embodiments, a nanoparticle comprises at least 0.1 mg/ml AMD3100 covalently conjugated to its surface. In some embodiments, a nanoparticle comprises at least 0.5 mg/ml AMD3100 covalently conjugated to its surface. In some embodiments, a nanoparticle comprises at least 0.625 mg/ml AMD3100 covalently conjugated to its surface. In some embodiments, a nanoparticle comprises at least 0.75 mg/ml AMD3100 covalently conjugated to its surface. In some embodiments, a nanoparticle comprises at least 1 mg/ml AMD3100 covalently conjugated to its surface. In some embodiments, a nanoparticle comprises at most 1.2 mg/ml AMD3100 covalently conjugated to its surface. In some embodiments, a nanoparticle comprises at most 1.25 mg/ml AMD3100 covalently conjugated to its surface. In some embodiments, a nanoparticle comprises at most 1.26 mg/ml AMD3100 covalently conjugated to its surface. In some embodiments, a nanoparticle comprises at most 1.3 mg/ml AMD3100 covalently conjugated to its surface. In some embodiments, a nanoparticle comprises at most 1.5 mg/ml AMD3100 covalently conjugated to its surface.

In some embodiments, the nanoparticle disclosed herein further comprises a drug. In some embodiments, the nanoparticle comprises an aqueous interior. In some embodiments, the nanoparticle comprises an aqueous solution surrounded by a lipid layer. In some embodiments, the aqueous interior comprises the drug. In some embodiments, the drug is in the aqueous interior of the nanoparticle. In some embodiments, the drug is dissolved in the aqueous solution within the nanoparticle.

In some embodiments, the drug is functional within a cell. In some embodiments, the drug interacts with an intracellular target. In some embodiments, the drug targets an intracellular target. In some embodiments, the target is a protein. In some embodiments, the target is a macromolecule. In some embodiments, the target is a nucleic acid molecule.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, circulating nucleic acids, fragments of genomic DNA or RNA, degraded nucleic acids, ribozymes, viral RNA or DNA, nucleic acids of infectios origin, amplification products, modified nucleic acids, plasmidical or organellar nucleic acids and artificial nucleic acids such as oligonucleotides.

In some embodiments, the cell comprises a cancer cell. In some embodiments, the drug is an anticancer drug. As used herein, the term “an anticancer drug”, and “antineoplastic drug”, are used interchangeably and refer to any drug that is effective in the treatment of malignant, or cancerous, disease. Anticancer drugs are well known in the art and any such drug may be employed within a composition of the invention. In some embodiments, the drug is an anti-multiple myeloma drug.

In some embodiments, the anticancer drug is a chemotherapeutic agent. In some embodiments, the chemotherapeutic agent is selected from selected from the group consisting of a crosslinking agent, a strand break agent, an alkylating agent, an anti-metabolite agent, a microtubule disruptor, a radiomimetic agent, a radiosensitizer, an intercalator, a DNA replication inhibitor, an anthracycline, an etoposide, and a topoisomerase II inhibitor. In some embodiments, the chemotherapeutic agent is selected from the group consisting of cisplatin, carboplatin, etoposide, oxaliplatin, rituximab or trastuzumab, mechlorethamine, cyclophosphamide, bleomycin, doxorubicin, daunorubicin, cytarabine, methotrexate, hydroxyurea, or a combination thereof.

In some embodiments, the anticancer drug comprises a proteasome inhibitor. As used herein, the term “proteasome inhibitor” encompass any molecule that inhibits the function of a proteasome. In some embodiments, the proteasome inhibitor is a protease inhibitor. In some embodiments, the proteasome inhibitor prevents degradation of a pro-apoptotic factor in the cell disclosed herein. In some embodiments, the proteasome inhibitor induces or increases death of the cell. In some embodiments, the proteasome inhibitor induces or increases programmed cell death of the cell.

In some embodiments, the proteasome inhibitor comprises a small molecule. In some embodiments, the proteasome inhibitor comprises an organic compound with molecular weight of below 1000 Daltons. In some embodiments, the proteasome inhibitor disclosed herein inhibits a serine protease in the cell. In some embodiments the proteasome inhibitor inhibits a 26S proteasome in the cell. In some embodiments, the proteasome inhibitor comprises bortezomib. In some embodiments, the proteasome inhibitor is bortezomib. Bortezomib (PS-34, CAS NO: 179324-69-7, IUPAC name: [(1R)-3-methyl-1-[[(2S)-3-phenyl-2-(pyrazine-2-carbonylamino)propanoyl]amino]butyl]boronic acid), is an FDA approved anti-cancer medication.

In some embodiments, the drug is present in the composition of the invention at a lower dose than the dose at which the free drug is administered. It will be understood by a skilled artisan that the nanoparticle of the invention targets the delivery of the drug and reduces the amount of drug that ends up at undesired locations. Since a greater percentage of the drug reaches the target a lower dose of the drug can be used. Similarly, as shown herein in FIG. 2F, use of the nanoparticle lowers the IC50 of the drug, again allowing for a lower dose to be used. In some embodiments, a lower dose is at least 10, 15, 20, 25, 30, 35, 40, 45, 50, 55, 60, 65, 70, 75, 80, 85, 90, 95, 100, 110, 120, 25, 130, 140, 150, 160, 170, 175, 180, 190, 200, 250, 300, 350, 400, 450, or 500% lower. Each possibility represents a separate embodiment of the invention. In some embodiments, a lower dose is at least 20% lower. In some embodiments, a lower dose is at least 50% lower. In some embodiments, the dose of the free drug is the dose at which the drug is administered to treat a disease. In some embodiments, the composition comprising the lower does is for use in the treating the disease. In some embodiments, the dose of the free drug is the minimum effective dose (MED) of the free drug. In some embodiments, the composition comprises at most 90, 85, 80, 75, 70, 65, 60, 55, 50, 45, 40, 35, 30, 25, 20, 15, 10, 5 or 1% of the MED. Each possibility represents a separate embodiment of the invention. In some embodiments, the composition comprises at most 80% of the MED. In some embodiments, the composition comprises at most 70% of the MED. In some embodiments, the composition comprises at most 50% of the MED.

In some embodiments, the aqueous interior of the nanoparticle comprises a basic pH. In some embodiments, the pH of the aqueous interior is in the range of between 7.0 to 12.0. In some embodiments, the pH of the aqueous interior is in the range of between 7.0 to 11.5, between 7.0 to 11.0, between 7.0 to 10.5, between 7.0 to 10.0, between 7.5 to 12.0, between 7.5 to 11.5, between 7.5 to 11.0, between 7.5 to 10.5, between 7.5 to 10.0, between 8.0 to 12.0, between 8.0 to 11.5, between 8.0 to 11.0, between 8.0 to 10.5, between 8.0 to 10.0, between 8.5 to 12.0, between 8.5 to 11.5, between 8.5 to 11.0, between 8.5 to 10.5, between 8.5 to 10.0, between 9.0 to 12.0, between 9.0 to 11.5, between 9.0 to 11.0, between 9.0 to 10.5, or between 9.0 to 10.0. Each embodiment represents a separate embodiment of the present invention. In some embodiments, aqueous interior of the nanoparticle comprises a pH of about 9.5.

In some embodiments, the nanoparticle comprises a bortezomib concentration in the range of between 0.1 mg/ml to 10 mg/ml. In some embodiments, the bortezomib concentration in the nanoparticle is between 0.5 mg/ml to 5 mg/ml, between 0.5 mg/ml to 4 mg/ml, between 0.5 mg/ml to 3 mg/ml, between 0.5 mg/ml to 2 mg/ml, between 0.6 mg/ml to 5 mg/ml, between 0.6 mg/ml to 4 mg/ml, between 0.6 mg/ml to 3 mg/ml, between 0.6 mg/ml to 2 mg/ml, between 0.7 mg/ml to 5 mg/ml, between 0.7 mg/ml to 4 mg/ml, between 0.7 mg/ml to 3 mg/ml, between 0.7 mg/ml to 2 mg/ml, between 0.8 mg/ml to 5 mg/ml, between 0.8 mg/ml to 4 mg/ml, between 0.8 mg/ml to 3 mg/ml, between 0.8 mg/ml to 2 mg/ml, between 0.9 mg/ml to 5 mg/ml, between 0.9 mg/ml to 4 mg/ml, between 0.9 mg/ml to 3 mg/ml, between 0.9 mg/ml to 2 mg/ml, between 0.5 mg/ml to 1.5 mg/ml, between 0.5 mg/ml to 1.4 mg/ml, between 0.5 mg/ml to 1.3 mg/ml, between 0.5 mg/ml to 1.2 mg/ml, between 0.5 mg/ml to 1.1 mg/ml, between 0.6 mg/ml to 1.5 mg/ml, between 0.6 mg/ml to 1.4 mg/ml, between 0.6 mg/ml to 1.3 mg/ml, between 0.6 mg/ml to 1.2 mg/ml, between 0.6 mg/ml to 1.1 mg/ml, between 0.7 mg/ml to 1.5 mg/ml, between 0.7 mg/ml to 1.4 mg/ml, between 0.7 mg/ml to 1.3 mg/ml, between 0.7 mg/ml to 1.2 mg/ml, between 0.7 mg/ml to 1.1 mg/ml, between 0.8 mg/ml to 1.5 mg/ml, between 0.8 mg/ml to 1.4 mg/ml, between 0.8 mg/ml to 1.3 mg/ml, between 0.8 mg/ml to 1.2 mg/ml, between 0.8 mg/ml to 1.1 mg/ml, between 0.9 mg/ml to 1.5 mg/ml, between 0.9 mg/ml to 1.4 mg/ml, between 0.9 mg/ml to 1.3 mg/ml, between 0.9 mg/ml to 1.2 mg/ml, or between 0.9 mg/ml to 1.1 mg/ml. Each embodiment represents a separate embodiment of the present invention. In some embodiments, the nanoparticle comprises a bortezomib concentration of about 1 mg/ml.

In some embodiments, the nanoparticle further comprises a fluorescent tag. In some embodiments, the fluorescent tag is covalently conjugated to a lipid. In some embodiments, the fluorescent tag is covalently conjugated to PE. In some embodiments, the fluorescent tag is covalently conjugated to DSPE. Examples for fluorescent tags are well known in the art. In one embodiment, the fluorescent tag comprises Cyanine5 (Cy5). In some embodiments, the nanoparticle comprises DSPE-Cy5.

### Compositions

By another aspect, there is provided a composition comprising a nanoparticle of the invention. In some embodiments, there is provided a pharmaceutical composition comprising a nanoparticle disclosed herein and a pharmaceutically acceptable carrier, excipient or adjuvant.

The term “pharmaceutically acceptable” means suitable for administration to a subject, e.g., a human. For example, the term “pharmaceutically acceptable” can mean approved by a regulatory agency of the Federal or a state government or listed in the U.S. Pharmacopeia or other generally recognized pharmacopeia for use in animals, and more particularly in humans.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the pharmaceutical composition is formulated for administration to a subject. In some embodiments, the subject is a mammal. In some embodiments, the mammal is a human. In some embodiments, the human is in need of the administration. In some embodiments, the pharmaceutical composition is formulated for systemic administration. In some embodiments, systemic administration is administration to the bloodstream of a subject (i.e., intravenous administration). In some embodiments, the pharmaceutical composition is formulated for intravenous administration. In some embodiments, the pharmaceutical composition is formulated for administration to the bloodstream.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, or intraperitoneal. In some embodiments, administering comprises systemic administration. In some embodiments, the administering is systemic administration. In some embodiments, the administering is intravenous administration.

The dosage administered will be dependent upon the age, health, and weight of the recipient, kind of concurrent treatment, if any, frequency of treatment, and the nature of the effect desired.

### Methods

By another aspect, there is provided a method for treating a CXCR4 positive cancer in a subject in need thereof.

By another aspect, there is provided a method for treating a bone disease in a subject in need thereof.

By another aspect, there is provided a method of targeting an agent to bone in a subject, the method comprising loading the agent to a nanoparticle of the invention to produce a loaded nanoparticle and administering the loaded nanoparticle to the subject, thereby targeting an agent to bone in a subject.

In some embodiments, the method comprises administering to the subject the nanoparticle disclosed herein. In some embodiments, the method comprises administering to the subject the pharmaceutical composition disclosed herein. In some embodiments, the method comprises administering to the subject an effective amount of the nanoparticle or the pharmaceutical composition. In some embodiments, effective is therapeutically effective. In some embodiments, effective is effective in treating cancer. In some embodiments, effective is effective in treating the bone disease.

As used herein, the term “subject” refers to any subject, including a mammalian subject, for whom therapy is desired, for example, a human. In some embodiments, the subject is a mammal. In some embodiments, the subject is a human subject. In some embodiments, the subject is in need of treatment. In some embodiments, the subject is treatable by administering a composition of the invention. In some embodiments, the subject suffers from cancer. In some embodiments, the cancer is a CXCR4 cancer. In some embodiments, the subject suffers from a bone disease.

In some embodiments, CXCR4 positive cancer comprises a metastatic cancer. In some embodiments, CXCR4 is involved in metastatic spread of cancer cells in the subject. In some embodiments, a CXCR4 level above a predetermined threshold is indicative of a CXCR4 positive cancer. In some embodiments, the threshold is the CXCR4 expression level in noncancerous cells. In some embodiments, the noncancerous cells are of the same cell type as the cancerous cells. In some embodiments, a CXCR4 level above a predetermined threshold is indicative of a metastatic spread of the cancer in the subject. In some embodiments, a CXCR4 level above a predetermined threshold is indicative of poor progression-free survival (PFS) or overall survival (OS) of the subject. In some embodiments, a CXCR4 level above a predetermined threshold comprises increased CXCR4 surface protein level on the cancer cell of the subject. In some embodiments, increased is as compared to noncancerous cells.

In some embodiments, the method further comprises a step of determining the expression level of CXCR4. In some embodiments, expression comprises secretion. In some embodiments, CXCR4 levels are within the cancer. In some embodiments, CXCR4 levels are within the tumor. In some embodiments, CXCR4 levels are within the tumor microenvironment (TME). In some embodiments, CXCR4 levels are within the organ niche in which the cancer cells reside. In some embodiments, an expression level of CXCR4 above a predetermined threshold, indicates the subject is suitable for treatment with the nanoparticle of the invention, a composition of the invention or by a method of the invention. In some embodiments, expression level any one of CXCR4 below or equal to a predetermined threshold, indicates the subject is not suitable for treatment with the nanoparticle of the invention, a composition of the invention or by a method of the invention.

In some embodiments, there is provided a method for selecting a subject being suitable for treatment with the composition of the invention, comprising the steps of: (a) determining the expression of CXCR4 above a predetermined threshold indicates the subject is suitable for treatment with the composition of the invention, and (b) administering to a subject determined to be suitable for treatment according to step (a) a therapeutically effective amount of a composition of the invention.

In some embodiments, the determining step is performed in the subject or in a sample derived or obtained from the subject. In some embodiments, the sample comprises any bodily fluid, cell, tissue, biopsy, organ, or a combination thereof, derived or obtained from the subject. In some embodiments, the determining step is performed in vivo, ex vivo, or in vitro. In some embodiments, the method further comprises obtaining a sample from the subject. In some embodiments, the sample comprises cancer cells. In some embodiments, the sample comprises bone cells. In some embodiments, the bone cells are bone marrow cells.

Methods and means for in vitro and/or ex vivo assays, as described herein, are common and would be apparent to one of ordinary skill in the art (e.g., flow cytometry of immunohistochemistry).

In some embodiments, CXCR4 positive cancer comprises a solid tumor. In some embodiments, a CXCR4 positive cancer comprises a CXCR4 positive myeloproliferative disorder. In some embodiments, CXCR4 positive cancer comprises a liquid tumor. In some embodiments, a liquid tumor comprises blood cancer, bone marrow cancer, lymph node cancer, or any combination thereof. In some embodiments, a blood cancer is a hematopoietic cancer. In some embodiments, the CXCR4 positive cancer is a bone marrow cancer. In some embodiments, a liquid tumor is selected from: leukemia, lymphoma, multiple myeloma, or any combination thereof. In some embodiments, CXCR4 positive cancer comprises at least one cancer type selected from: hematopoietic cancer, melanoma, breast cancer, colorectal cancer, esophageal cancer, head and neck cancer, renal cancer, lung cancer, gynecologic cancer, pancreatic cancer prostate cancer, gallbladder cancer, liver cancer, or any combination thereof. In some embodiments, CXCR4 positive cancer comprises a hematopoietic cancer. In some embodiments, the blood cancer is polycythemia vera. In some embodiments, the myeloproliferative disorder is polycythemia vera. In some embodiments, the hematopoietic cancer is selected from: multiple myeloma, leukemia, lymphoma, or any combination thereof. In some embodiments, the hematopoietic cancer comprises multiple myeloma. In some embodiments, the CXCR4 cancer is multiple myeloma. In some embodiments, the CXCR4 cancer is leukemia. In some embodiments, the leukemia is a myeloid leukemia. In some embodiments, the myeloid leukemia is acute myeloid leukemia (AML). In some embodiments, the AML is acute promyelocytic leukemia (APML). In some embodiments, the CXCR4 positive cancer is a bone cancer. In some embodiments, the bone cancer is an osteosarcoma. In some embodiments, the bone cancer is a cancer that metastasizes to the bone. In some embodiments, the bone cancer is a bone metastasis. Bone metastasis are known to occur from a wide variety of tissues including for example from lung, breast, prostate and others.

In some embodiments, the bone disease is a bone marrow disease. In some embodiments, bone is bone marrow. In some embodiments, the bone disease is selected from bone cancer, metastasis to the bone, osteoporosis, Paget's disease of bone, rickets, osteopenia, polycythemia vera and osteogenesis imperfecta. In some embodiments, the bone disease is bone cancer. In some embodiments, the bone cancer is a CXCR4 positive cancer. In some embodiments, the bone cancer is a leukemia. In some embodiments, the bone cancer is multiple myeloma. In some embodiments, the bone cancer is bone metastasis. In some embodiments, the bone cancer is bone metastasis of a CXCR4 negative cancer. In some embodiments, the bone cancer is bone metastasis of a CXCR4 positive cancer.

In some embodiments, the dose of bortezomib is equivalent to a mouse dose of about 1 mg/kg body weight. In some embodiments, the dose of bortezomib is equivalent to a mouse dose of about 3 mg/kg body weight. In some embodiments, the dose of bortezomib is equivalent to a mouse dose of between 1 and 3 mg/kg body weight. In some embodiments, a human dose is about 1/12 of the mouse dose. In some embodiments, the mouse dose is converted to the human dose by dividing by 12.3. In some embodiments, the human dose of bortezomib is about 0.0813 mg/kg body weight. In some embodiments, the human dose of bortezomib is about 0.244 mg/kg body weight. In some embodiments, the human dose of bortezomib is between 0.0813 and 0.244 mg/kg body weight.

In some embodiments, the method further comprises confirming expression of CXCR4 in the cancer. In some embodiments, the method further comprises confirming expression of CXCR4 in cells of the cancer. In some embodiments, expression is mRNA expression. In some embodiments, expression is protein expression. In some embodiments, expression is surface expression. In some embodiments, expression is expression on a surface of the cancer cells. In some embodiments, the administering is to a subject with confirmed expression of CXCR4 in their cancer.

In some embodiments, confirming comprises receiving a sample from the subject. In some embodiments, the sample comprises cells. In some embodiments, the sample comprises cancer cells. In some embodiments, the sample is a tumor sample. In some embodiments, the sample is a biopsy. In some embodiments, the sample comprises tissue. In some embodiments, the sample comprises a bodily fluid. In some embodiments, the bodily fluid is selected from at least one of: blood, serum, plasma, gastric fluid, intestinal fluid, saliva, bile, tumor fluid, breast milk, urine, interstitial fluid, cerebral spinal fluid, tumor fluid and stool. In some embodiments, the sample is a blood sample. In some embodiments, blood is peripheral blood. In some embodiments, the sample is a bone marrow sample.

In some embodiments, the method further comprises measuring expression of CXCR4 in the sample. In some embodiments, in the sample is in cells of the sample. In some embodiments, the cells are cancer cells. In some embodiments, in the cells is on the surface of the cells. Methods of measuring expression, and in particular surface protein expression are well known in the art and any such method may be used. Such methods include, but are not limited to flow cytometry, western blotting, immunostaining, PCR, sequencing, northern blot, and microarrays. In some embodiments, surface protein expression is determined by flow cytometry.

In some embodiments, expression is any expression. In some embodiments, expression is positive expression. In some embodiments, expression is expression above a predetermined threshold. In some embodiments, the threshold is zero expression. In some embodiments, the threshold is the expression level in healthy cells. In some embodiments, the healthy cells are from the same cell type as the cancerous cells. In some embodiments, expression indicates the subject is suitable for the administering. In some embodiments, expression above the predetermined threshold indicates the subject is suitable for the administering. In some embodiments, expression indicates the subject is confirmed to have CXCR4 positive cancer. In some embodiments, expression above the predetermined threshold indicates the subject is confirmed to have CXCR4 positive cancer.

In some embodiments, the loading is active loading. In some embodiments, the loading is passive loading. In some embodiments, the loading is via a concentration gradient. In some embodiments, the loading is via a pH gradient. In some embodiments, the concentration gradient is concentration of a sugar. In some embodiments, the sugar is a sugar alcohol. In some embodiments, the sugar alcohol is mannitol. In some embodiments, the concentration gradient is a gradient of a sugar and an acid. In some embodiments, the concentration gradient is a gradient of mannitol and an acid. In some embodiments, the acid is acetic acid.

In some embodiments, the agent is a therapeutic agent. In some embodiments, the agent is a drug. In some embodiments, the agent is a protein. In some embodiments, the agent is a therapeutic. In some embodiments, the agent is loaded into the nanoparticle. In some embodiments, the agent is loaded into the core of the nanoparticle. In some embodiments, the agent is loaded onto the nanoparticle. In some embodiments, the agent is loaded into the shell of the nanoparticle. In some embodiments, the agent is loaded onto the surface of the nanoparticle.

In some embodiments, targeting is delivering. In some embodiments, the method is a method of delivering the agent to the bone of the subject. In some embodiments, to the bone is to a bone cell. In some embodiments, the delivering is to a bone cell. In some embodiments, the delivering is to a CXCR4 positive bone cell. In some embodiments, the delivering is to the surface of the cell. In some embodiments, the delivering is to the inside of the bone cell. In some embodiments, the inside is the cytosol.

In some embodiments, the administering is systemic administering. In some embodiments, the systemic administering is intravenous administering. It will be understood by a skilled artisan that systemic administration and in particular IV administration requires the loaded nanoparticle to pass through the blood stream in order to reach the bone. As demonstrated hereinbelow, covalent linking of the AMD to the nanoparticle is essential to facilitate systemic targeting to the bone. If a weaker form of linking is used (e.g., electrostatic adsorption), the AMD is lost in the blood and targeting to the bone does not occur.

By another aspect, there is provided a method of determining suitability of a subject to be treated by a method of the invention, the method comprising receiving a sample from the subject and measuring expression of CXCR4 in the sample, wherein expression of CXCR4 indicates the subject is suitable to be treated by a method of the invention.

In some embodiments, measuring expression in the sample is measuring expression is cells of the sample. In some embodiments, the cells are cancer cells. In some embodiments, in the cells is on a surface of the cells. In some embodiments, the surface is the plasma membrane. In some embodiments, expression is expression above a predetermined threshold. In some embodiments, expression above a predetermined threshold indicates the subject is suitable to be treated. In some embodiments, expression at a predetermined threshold indicates the subject is suitable to be treated. In some embodiments, expression at a predetermined threshold indicates the subject is not suitable to be treated. In some embodiments, expression below a predetermined threshold indicates the subject is not suitable to be treated.

In some embodiments, the method further comprises treating a subject determined to be suitable. In some embodiments, treating is by a method of the invention. In some embodiments, the method further comprises administering an alternative treatment to a subject determined to be unsuitable. In some embodiments, the alternative treatment is an anticancer treatment. In some embodiments, an anticancer treatment comprises administering an anticancer agent. Examples of alternative treatments include, but are not limited to, chemotherapy, radiation therapy, surgery, immunotherapy, and targeted therapy. Chemotherapeutic agents are well known in the art and any chemotherapy may be used. Similarly, immunotherapies such as immune checkpoint inhibitors, CAR-T/NK administration and anticancer vaccines are also well known, as are targeted therapies such as antibodies against specific pro-tumorigenic or pro-metastatic proteins. Any such alternative therapy may be used to treat a subject determined to be unsuitable.

By another aspect, there is provided a method of covalently linking a molecule comprising a secondary amine to a lipid nanoparticle, the method comprising:
a. providing a lipid nanoparticle comprising at least one carboxyl group;
b. reacting the provided lipid nanoparticle with sulfo-N-hydroxysulfosuccinimide (S-NHS) and a carbodiimide coupling agent to obtain S-NHS active ester and,
c. reacting the S-NHS active ester with a molecule comprising a secondary amine;
   thereby covalently linking a secondary amine to a lipid nanoparticle.

In some embodiments, the method is for forming an amide bond between the secondary amine and the carboxyl group of the lipid nanoparticle. In some embodiments, the method comprises forming an amide bond between the secondary amine and the carboxyl group of the lipid nanoparticle. In some embodiments, the secondary amine is a tertiary amide upon conjugation. In some embodiments, the secondary amine is a tertiary amide upon linking. In some embodiments, linking is conjugation. In some embodiments, linking is conjugating.

In some embodiments, the lipid nanoparticle comprises PEG. In some embodiments, the lipid nanoparticle comprises PEG-lipid. In some embodiments, the carboxyl group is covalently bound to PEG-lipid. In some embodiments, the providing comprises providing a lipid nanoparticle and generating a carboxyl group on the lipid nanoparticle. In some embodiments, generating comprises linking. In some embodiments, linking is covalently linking. In some embodiments, PEG-lipid comprises PEG-DSPE. In some embodiments, the carbodiimide coupling agent is selected from: (1-ethyl-3-(3-dimethylaminopropyl)carbodiimide hydrochloride) EDC, dicyclohexylcarbodiimide (DCC) diisopropylcarbodiimide (DIC), or any combination thereof. In some embodiments, the carbodiimide coupling agent comprises EDC.

By another aspect, there is provided a method of producing a therapeutic nanoparticle suitable for treating a CXCR4 positive cancer or a bone disease, the method comprising, selecting a therapeutic agent that treats the CXCR4 positive cancer or bone disease and loading the selected agent into a nanoparticle of the invention, thereby producing a therapeutic nanoparticle.

A skilled artisan will be able to select a therapeutic agent which can be used for treating a specific disease. The agents for treating various cancer and bone diseases are well known in the art and a skilled artisan would be able to make such a selection. In some embodiments, the method further comprises producing the nanoparticle by a method of covalent linking of the invention. In some embodiments, the nanoparticle of the invention is for use in treating a CXCR4 positive cancer. In some embodiments, the produced therapeutic nanoparticle is for use in treating a CXCR4 positive cancer. In some embodiments, the nanoparticle of the invention is for use in treating a bone disease. In some embodiments, the produced therapeutic nanoparticle is for use in treating a bone disease. In some embodiments, the nanoparticle of the invention is for use in the production of a medicament for treating a CXCR4 positive cancer. In some embodiments, the produced therapeutic nanoparticle is for use in the production of a medicament for treating a CXCR4 positive cancer. In some embodiments, the nanoparticle of the invention is for use in the production of a medicament for treating a bone disease. In some embodiments, the produced therapeutic nanoparticle is for use in the production of a medicament for treating a bone disease.

### General

The term “expression” as used herein refers to the biosynthesis of a gene product, including the transcription and/or translation of the gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Small molecules, or micromolecules, are well known in the art and refer to low molecular weight (≤1000 Daltons) organic compounds that can regulate a biological process or be used as a therapy. In some embodiments, a small molecule is a therapeutic agent. In some embodiments, the nanoparticle is a therapeutic agent.

As used herein, the term “contacting” refers to at least one of: incubating, mixing, centrifuging or any combination thereof.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Cell Culture

CAG, U266 and RPMI8226 human MM cells; (American Type Culture Collection; ATCC); KMS-11 (originally from the Japanese Collection of Research Bioresource); human MM cells and MPC-11 murine myeloma cell line (kindly provided by Professor Ralph Sanderson, University of Alabama, USA) and HL60 human acute myeloid leukemia (ATCC) were used. Cells were cultured in RPMI-1640 medium supplemented with 10% fetal bovine serum (FBS), supplemented with 1% L-glutamine, 1% sodium pyruvate and 1% pen-strep-neomycin. All cells were cultured at 37° C. in 5% CO2 for no more than 6 months after being thawed from the authentic stocks.

### Liposomes Preparation and Characterization

### AMD Targeted Liposomes

Liposomes were prepared using the ethanol injection method. First, the lipids were weighted, HSPC:Cholesterol:DSPE-PEG1000-COOH (The carboxyl group is serving as a binding site for the targeting molecule), at a molar ratio of 55:40:5 and then they were dissolved in absolute ethanol and warmed in a temperature that is higher than the Tm of the lipids (65° C.). In some experiments, distearoylphosphatidylcholine-Cyanine5 (DSPE-Cy5) were also incorporated into the lipid mixture to form fluorescent liposomes. When the lipids were fully dissolved in ethanol, the whole suspension were injected to an aqueous solution acetic acid (150 mM) and mannitol (150 mM) adjusted to pH=9 using the ethanol injection method22. The dispersion (MLV) was then passed by extrusion to obtain homogenous liposomes with a size of ~100 nm. Liposomes were dialyzed against 50 mM MBS pH=6 at room temperature for 24 h (three times buffer replacement) using dialysis bags with a molecular weight cut-off of 12-14 kDa.

The conjugation process between the AMD molecules to the phospholipid backbone of the liposomes surface were applied through a nucleophilic substitution carbodiimide crosslinking reaction with the reagents-(3-dimethylaminopropyl)-3-ethylcarbodimide hydrochloride (EDC) and sulfo-N-hydroxy succinimide (S-NHS) to form an amide linkage. For validation of this process, we used TLC method, silica gel served as stationary phase and chloroform:methanol:ammonia hydroxide 25% in H2O at a ratio of 5:12.5:3 as the mobile phase.

### AMD Electrostatic Conjugation to Bortezomib Liposomes

Liposomes were prepared using the ethanol injection method. First, the lipids were weighted, HSPC:Cholesterol at a molar ratio of 70:30 (taken from Ullah et al.) or DPPA: HSPC:Cholesterol:DSPE-PEG 1000 at a molar ratio of 10:45:40:5 (the liposome of the invention with DPPA added to allow electrostatic conjugation). Subsequently, they were dissolved in absolute ethanol and warmed in a temperature that is higher than the Tm of the lipids (65° C.). In some experiments, Rhodamine was also incorporated into the lipid mixture to form fluorescent liposomes. When the lipids were fully dissolved in ethanol, the whole suspension was injected to an aqueous solution acetic acid (150 mM) and mannitol (150 mM) adjusted to pH=9 using the ethanol injection method. The dispersion (MLV) was then passed by extrusion to obtain homogenous liposomes with a size of ~100 nm. Liposomes were dialyzed against 50 mM MBS pH=6 at room temperature for 24 h (three times buffer replacement) using dialysis bags with a molecular weight cut-off of 12-14 kDa. The conjugation process between the AMD molecules to the phospholipid backbone of the liposomes surface were applied through electrostatic binding. For validation of this process, we used TLC method, silica gel served as stationary phase and chloroform:methanol:ammonia hydroxide 25% in H2O at a ratio of 5:12.5:3 as the mobile phase. In some experiments, Zeta potential was evaluated using dynamic light scattering (DLS).

### Encapsulation of Bortezomib Into Liposomes

Active loading methods was used by chemical trapping method through a concentration gradient of mannitol and acetic acid, where the concentration of mannitol and acetic acid is higher in the liposomal core compared to the external medium to form boronic acid moiety to covalently bind to mannitol, forming boronated esters that increase the loading rate of bortezomib in the liposomal core16,17. In addition, to further increase the encapsulation efficiency and allow the reaction to occur only inside the liposome, we created a transmembrane pH gradient between the exterior (pH=6.5) and the interior (pH=9) environments of the liposomes. 18 Bortezomib was loaded into the liposomes by overnight incubation of liposomal formulation with bortezomib (2, 2.5 or 3 mg/ml pH 6.5). Then, liposomes were dialyzed against PBS (pH=7) at room temperature for 24 h (three times buffer replacement). Then liposomes were burst using DMSO and the concentration of the loaded drug was measured using microplate reader at a wavelength of 325 nm. In addition, the particle size distribution and concentration were measured using dynamic light scattering (DLS).

### Transwell Migration Assay

Migration was determined by using the transwell migration assay according to the manufacturer's instructions and as previously described. CAG Cells were incubated with 1% FBS and were treated with ATBL, bortezomib liposomes, AMD or vehicle, for 90 min. Then, 0.5×105 cells were added to the upper compartment of the chamber. The lower compartment was filled with 1% FCS RPMI medium with 30 nM SDF-1. After 24 h, cells that migrated to the bottom were collected and counted using cell counting chamber.

### Modified Boyden Chamber Assay

Migration of MM cells, as indicated in the text, were assessed by the modified Boyden chamber assay, as previously described. Briefly, RPMI8226 and CAG cells, 1×106 cells in 0.2 ml RPMI medium, were added to a Boyden chamber filter which has been coated whit 100 μl fibronectin (10 mg/ml) for migration testing. The lower compartment was filled with medium containing 10% plasma obtained from vehicle-treated mice, bortezomib-treated mice (at the 24 h time-point) or ATBL-treated mice at 24, 72 and 168 h time-point. After 24 h, the cells that migrated to the bottom were collected and counted by flow cytometry, using 7.3 μm counting beads (Bangs Laboratories, Fishers, IN, USA), according to the manufacturer's instructions.

### Flow Cytometry for Determination of CXCR4 Expression

To determine the expression of CXCR4 on the surface of several MM cell lines and HL-60 cells, cells were immunostained with anti-human CD184 for CXCR4 receptor, and then the cells were subjected to LSR Fortessa flow cytometer (BD) and analyzed with FlowJo 10.

### Apoptosis Assay

CAG and RPMI8226 cell were obtained from cultures and replated (250,000 cells/well in a 96-well plate) in RPMI medium and treated with ATBL, bortezomib liposomes, bortezomib (0.1 mg/ml), empty liposomes (10%), AMD3100 (50 μM) or vehicle control for 48 h. In another experiment cells were incubated with 10% plasma obtained from vehicle-treated mice, bortezomib-treated mice (at the 24 h time-point) or ATBL-treated mice at 24, 72 and 168 h time-point post administration. Then cell viability and apoptosis status; healthy, early apoptosis, late apoptosis and dead, were determent using 7AAD and Annexin V staining and were used in accordance with the manufacturer's instruction. At least 50,000 events were acquired using a LSRFortessa flow cytometer (BD) and analyzed with FlowJo 10.

### Cell Cycle Analysis

RPMI 8226 cells kept in serum-free medium for 24 h for synchronization were then cultured in the presence of 10% plasma obtained from vehicle-treated mice, bortezomib-treated mice (at the 24 h time-point) or ATBL-treated mice (at 24, 72 and 168 h time-point) for evaluation of the cell cycle, as previously described.

For flow cytometry analysis, RPMI 8226 cells were fixed with 70% ethanol for at least 1 h at 4° C. The cells were washed in PBS and stained with 40 ug/ml propidium iodide for the evaluation of DNA content and analyzed for cell cycle status with LSRFortessa flow cytometer (BD) and analyzed with FlowJo 10.

### Expression of Luciferase in RPMI8226 Cells

We used RPMI8226 derived cell line and infect the cells with lentivirus directing expression of firefly luciferase. The cDNA encoding firefly luciferase was previously subcloned into the pLenti6-V5/Dest plasmid (InvitroGen)26. This plasmid was used in order to generate lentivirus. Lentiviruses directing expression of this cDNA was produced in HEK293FT cells and used to infect target RPMI8226 cells. Cells stably expressing luciferase was then selected using cell sorting for GFP+ cells.

### Animal Tumor Models and Drugs

5-6 weeks old SCID female mice were underwent whole-body radiation at a total dose of 250 rads (Department of Radiotherapy, RHCC, Haifa). After 24 h, the mice were intravenously injected with RPMI 8226 cells (5×106) or HL-60 cells (5×106). After 2-3 weeks, where sufficient tumor growth was detected by bioluminescence IVIS imaging, different treatments were initiated. The treatments were given once a week for another 40-60 days, or as indicated in the figure. Tumor volume was measured ones a week. Mice were followed up daily and when mice were showing signs of paralysis they were sacrificed.

Detection of Tumor Progression by Bioluminescence Imaging Mice were injected with 75 mg/kg luciferin and imaged for bioluminescence. The home-built bioluminescence system used an electron multiplying CCD (Andor Technology, Belfast, United Kingdom) with an exposure time of 15 seconds, an electron multiplication gain of 500-voltage gain-200, 5-by-5 binning, and background subtraction. Images were analyzed with the use of ImageJ software (National Institutes of Health, Bethesda, MD).

### Histology

### Biodistribution and Quantification of Liposomes Delivery Into Tumors

In order to quantitatively track the liposomes' bio-distribution in vivo, the liposomes are Gd entrapped and are tagged with Cy5. CAG and RPMI8226 cells tagged with luciferase are IV injected as described herein for the tumor models. Then mice are treated whit ATBL, bortezomib liposomes and Gd, that serves as a control, twice a week for a period of 20-30 days. Then the expression of luciferase and Cy5 in mice at 4, 8, 12, 24 and 48 h is measured using IVIS to determine tumor development and liposomes spread. At the end point, mice are sacrificed and different samples from various organs are removed. The samples are than scanned using the coupled plasma atomic emission spectroscopy (ICP-AES) to determent the amount of GD in the organs.

### Biodistribution and Quantification of Liposomes Delivery Into the Bone Marrow

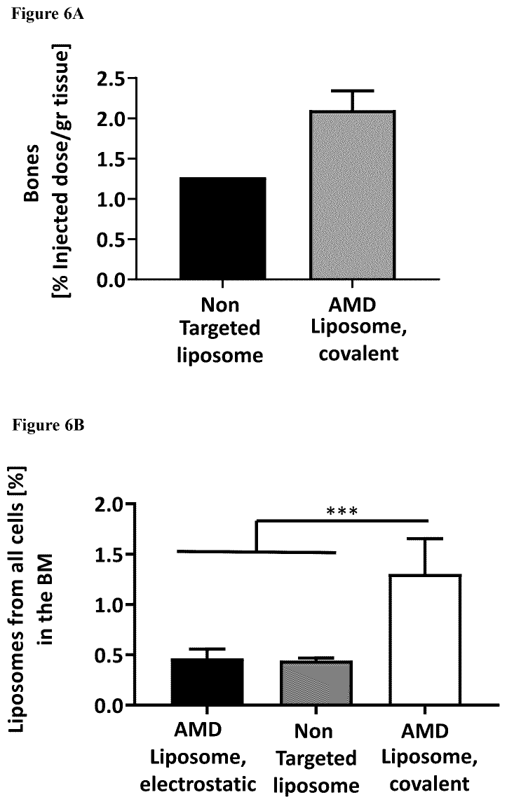

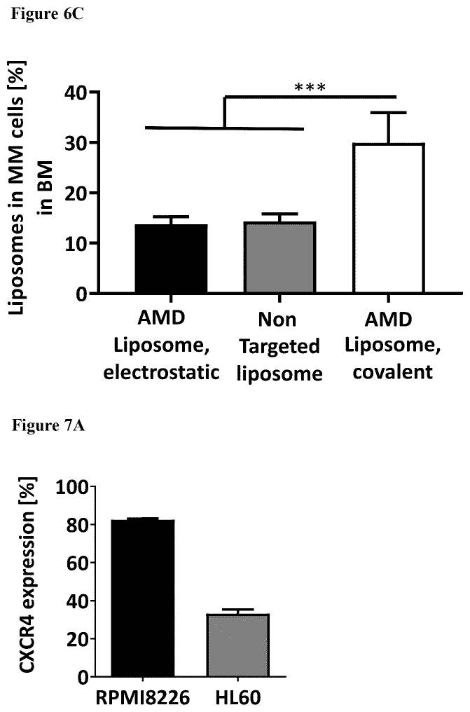

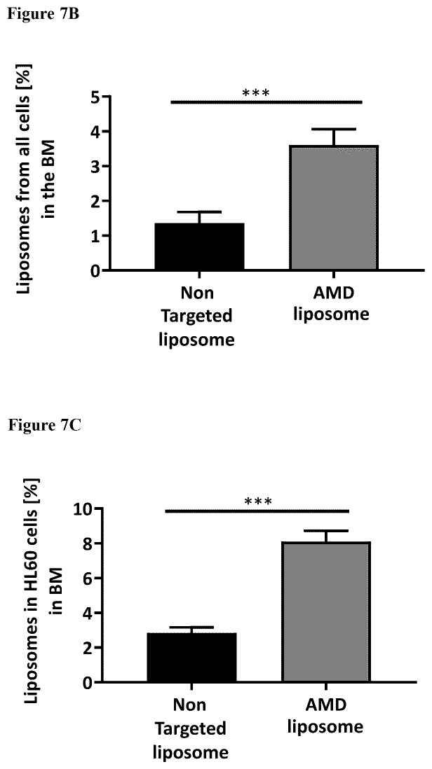

In order to quantitatively track the liposomes' bio-distribution in vivo, the liposomes are Gd entrapped and are tagged with rhodamine. RPMI8226 or HL-60 cells tagged with luciferase are IV injected as described herein for the tumor models. Then mice are treated with AMD-covalently or electrostatically bound to liposomes and non-targeted liposomes tagged with rhodamine (FIG. 6B-6C and 7B-7C). After 24 hours (from injection), mice were sacrificed bones were flashed and evaluated for rhodamine positive cells using flow cytometry. In another experiment, (FIG. 6A), the same liposomes containing gadolinium were injected to mice and 24 hours later gadolinium was assessed in the bones using the ICP.

Statistical analysis

Data is expressed as mean±standard deviation (SD). The statistical significance of differences was assessed by one-way ANOVA, followed by Newman-Keuls ad hoc statistical test using GraphPad Prism 5 software (La Jolla, CA). Differences between all groups were compared with each other and considered significant at values below 0.05.

### Example 1: Generation and Chemical Properties of AMD3100 Targeting Bortezomib Liposome (ATBL)

Resistance to bortezomib and dose-limiting toxicities are significant barriers to the treatment of multiple myeloma (MM). The bone marrow (BM) niche plays a major role in MM bortezomib resistance. Drug delivery with targeted liposomes has been shown to improve specificity and efficacy and reduce toxicity. The inventors aimed to encapsulate bortezomib in a liposome and to conjugate AMD3100 to the surface of the liposome, in the purpose of targeting CXCR4 expressed by hematopoietic stem cells (HSCs) and MM cells.

PEGylated liposomes were constructed by hydrogenated soybean phosphatidylcholine (HSPC), cholesterol and 1,2-Distearoyl-sn-glycero-3-phosphorylethanolamine (DSPE)-polyethylene glycol (PEG)1000-COOH at a total lipid concentration of 100 mM, in molar ratios of 55:40:5, using the ethanol injection method. This composition was chosen because it is very stable and already FDA approved. During nanoparticle fabrication, a carboxyl group was attached to DSPE-PEG1000 for further conjugation of the AMD3100 molecule to form the AMD liposome. In addition, DSPE-Cy5 was incorporated into the nanoparticles as a fluorescent labeled lipid to allow tracking of the liposomes. Then, the liposomes were extruded to reduce their size to 107±30 nm, as measured by DLS (FIG. 1D). Next, the inventors developed the AMD liposomes to target the CXCR4 receptor on MM cells. The AMD3100 molecule was attached to the surface of the prepared liposomes by EDC carbodiimide crosslinking reaction with S-NHS. Initially an attempt was made to use NHS however it was found to have a high sensitivity to water and S-NHS was found to be significantly more stable. A scheme illustrating the series of reactions of AMD liposomes construction is shown in FIG. 1A. In order to evaluate the successful formation of AMD liposomes, a thin-layer chromatography (TLC) was performed.

As the goal was to develop encapsulation for bortezomib therapy along with a targeted moiety, bortezomib was encapsulated into the AMD liposomes. Passive and active loading methods were performed to search for the most efficient encapsulation method. It was found that active loading had a significantly higher encapsulation efficacy compared to passive loading (FIG. 1B). Thus, it was chosen to continue the studies using the active loading method.

To further increase the active encapsulation efficiency, several strategies were employed. First, since bortezomib is a small molecule and can simply pass across the lipid bilayer of liposomes, the inventors used a chemical trapping method through a concentration gradient of mannitol, where the concentration of mannitol is higher in the liposomal core compared to the external medium. Second, acetic acid was added to enhance the encapsulation efficiency of bortezomib. These two strategies allowed boronic acid moiety to be able to covalently bind to mannitol, forming boronated esters that increase the loading rate of bortezomib in the liposomal core. In addition, to further increase the encapsulation efficiency and allow the reaction to occur only inside the liposomes, it was necessary to create a transmembrane pH gradient between the exterior (pH=6.5) and the interior (pH=9.5) environments of the liposomes. A schematic illustration of these bortezomib liposomes encapsulation is shown in FIG. 1C. Moreover, to quantify the concentration of bortezomib encapsulated, an absorbance scan was measured over a range of wavelengths (230-1000 nm) to identify the optimal wavelength for detection using microplate readers. The highest peak was observed at 325 nm, and calibration curve of known bortezomib concentrations was measured in order to verify that a straight line is indeed obtained and that the selected wavelength is suitable. Next, to test the effect of bortezomib concentration on the encapsulation efficiency (% EE), bortezomib was added at increasing concentrations of 2, 2.5 and 3 mg/ml for active loading. The results show that a significant higher % EE was observed when liposomes were incubated with 2 mg/ml (49.5%) compared to 2.5 and 3 mg/ml (43.6% and 27.3% respectively) of bortezomib (FIG. 1E). The inventors next sought to determine the drug release profile of bortezomib liposomes. Drug release rate is correlated with toxicity and inversely correlated with the therapeutic activity of liposomes in cancer treatment, making the release of a drug to be very critical in liposomal drug pharmacodynamics. Drug release profile of bortezomib through liposomes at 37° C. was measured for 10 days. Bortezomib release was approximately 15% within these 10 days (FIG. 1F). The lower release observed indicates that this formulation is stable and can be explained by the construction of lipids in the liposomes. Overall, these results demonstrate that the methods presented above enable generation of an AMD3100 targeting bortezomib liposome (ATBL) with high % EE and stability.

### Example 2: ATBL Actively Targets MM Cells Through CXCR4

To test the ability of the targeting liposomes to bind to CXCR4 on MM cells, a migration assay was performed using the Boyden chamber assay. Since it was found that RPMI8226 cells secrete abundant levels of SDF-1, the inventors further analyzed CAG cells. To this end, CAG cells were treated with AMD liposomes, AMD, empty liposomes and vehicle. Subsequently, the cells were added to the upper chamber and the lower compartment was filled with SDF-1. After 24 h, cells that migrated to the bottom compartment were collected and counted. It was found that AMD liposomes significantly inhibited CAG cell migration in response to SDF-1, compared to AMD, empty liposomes, and vehicle (FIG. 2A).

Next, the uptake kinetics of the liposomes by CAG and RPMI8226 MM cells was evaluated using flow cytometry. CAG and RPMI8226 cell lines were used since it was found that they display higher CXCR4 expression compared with the other cell lines tested. To this end, the cells were incubated with media supplemented with 1% or 3% of AMD liposomes or non-targeted liposomes labeled with Cy 5 for 30, 60 and 90 min. The results show that the cellular uptake of AMD liposomes at 90 min was greater than empty liposomes, reaching 1.9-and 1.3-fold increase in both 1% and 3% liposomes concentrations, respectively, in the RPMI 8226 cells. Similarly, and to a higher magnitude, AMD liposomes displayed 93.5-and 71.9-fold increase in liposome uptake when using CAG cells compared to empty liposomes at 1% and 3%, respectively (FIGS. 2B and 2C). These results indicate that AMD binding to the liposomes not only serves as a targeting molecule but actually increases the cellular uptake of the liposomes.

To determine the therapeutic effect of ATBL compared to free bortezomib, a cell viability and apoptosis assay was used. To this end, CAG and RPMI8226 cells were treated with ATBL, bortezomib in naked liposomes, free bortezomib, AMD on empty liposomes, free AMD, empty naked liposomes and vehicle. The results in FIGS. 2D and 2E show that ATBL, bortezomib liposomes and bortezomib induce a significant higher late apoptosis compared to other treatments tested, while no significant differences in early apoptosis and viability were observed in both CAG and RPMI8226 cells (data not shown). It should be noted that while no significant difference in late apoptosis between ATBL, bortezomib liposomes and bortezomib, was detected, the fact that the same therapeutic effect was observed, indicates that at least in vitro, ATBL is an effective treatment molecule. However, the inventors hypothesized that in an in vivo model, ATBL will have better therapeutic efficacy due to its targeting properties.

The half maximal inhibitory concentration (IC50) of bortezomib and ATBL was evaluated in RPMI8226 cells. The IC50 of bortezomib (11 nM) was almost 2-fold more then ATBL (5.96nM), indicating the higher potency of ATBL (FIG. 2F).

### Example 3: ATBL Treatment of CXCR4 Positive Cancer

For an efficacy study, RPMI8226 cells tagged with luciferase were injected IV into the tail vein of SCID mice and tumor growth and expansion was monitored. After 2-3 weeks, where sufficient tumor progression was detected, treatments with ATBL, free AMD and free bortezomib in combination (mixed but without liposomes) or vehicle were initiated. The different treatments were given once a week for a period of 20-30 days and tumor progression was assessed once a week by IVIS in vivo imaging. Mice treated with free AMD and free bortezomib in combination showed a modest but not statistically significant decrease in tumor burden. However, ATBL not only produced a significant decrease in tumor burden compared with the control, but surprisingly this decrease was also significant when compared with free AMD and free bortezomib treatment (FIG. 3). This indicates the significantly improved therapeutic potential of the developed ATBL therapy, as compared to bortezomib therapy alone, AMD3100 therapy alone, and even when treating with these agents is combined.

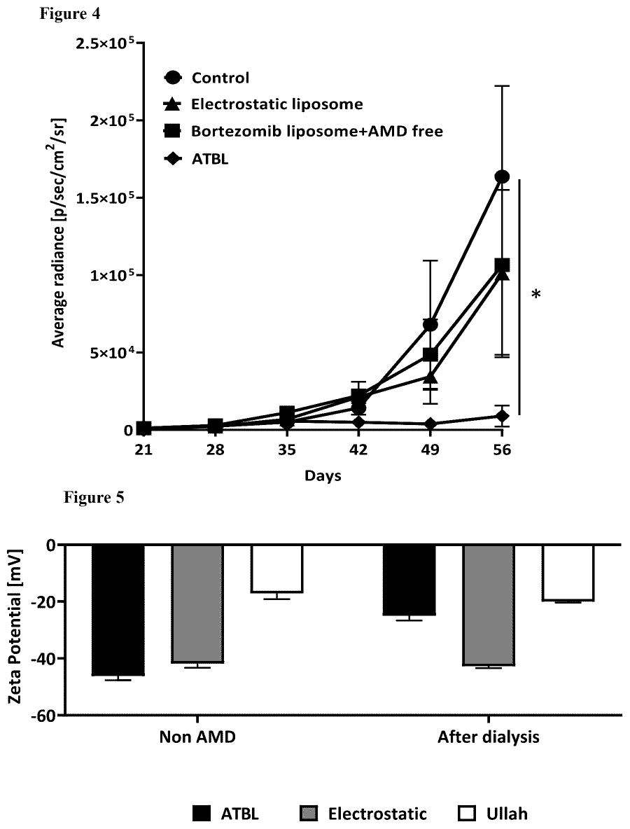

In a similar experiment, RPMI8226 cells were used as a tumor model (5×106 cells/mouse) as before. On day 28 after IV injection of the cancer cells treatment was initiated with ATBL, bortezomib loaded naked liposome in combination with free AMD and bortezomib loaded liposomes to which AMD was electrostatically adhered to the surface of the liposome. Ullah et al., “CXCR4-targeted liposomal mediated co-delivery of pirfenidone and AMD3100 for the treatment of TGFB-induced HSC-T6 cells activation”, Int. Jour. Of Nanomedicine, 2019, 14:2927-2944, the contents of which are hereby incorporated by reference in their entirety, demonstrated liposomes which had AMD adsorbed to their surface by electrostatic interaction of the positively charged AMD and negatively charged liposome. Thus, this method for attaching AMD to the liposome surface was also tested. As before tumor growth was assessed by the bioluminescence IVIS system. Treatment with either the naked liposome in combination with free AMD or the liposome with AMD electrostatically adhered produced a small decrease in tumor volume (FIG. 4). In contrast ATBL nearly completely inhibited tumor growth, producing a substantial decrease as compared to control and both of the other liposome compositions. This indicates that covalent bonding of AMD to the liposome surface produces a greatly superior therapeutic. Indeed, the composition with free AMD and the composition with electrostatically adhered AMD produced very similar effects suggesting that the electrostatically adhered AMD dissociates in the blood and does not actually produce targeting to CXCR4. In contrast, the AMD in ATBL is covalently bonded to the liposome and cannot dissociate in the blood, allowing for CXCR4 targeting and more effective cancer cell killing.

To test this hypothesis, three different AMD liposome compositions were produced. The first was ATBL made by covalently binding the AMD to the liposome surface. The second was the liposomes taught in Ullah et al. These liposomes were made of 70% HSPC and 30% cholesterol with AMD adsorbed to the surface electrostatically. The third was liposomes with the lipid makeup of the ATBL liposomes (HSPC, cholesterol and DSPE-PEG100) supplemented with diphenylphosphoryl azide (DPPA) to ensure the liposome has a strong negative charge and with the AMD adsorbed to the surface electrostatically as done by Ullah. All liposomes were produced with an AMD starting concentration of 10 mg/ml such that it was greatly in excess. The zeta potential of the liposomes was measured without AMD and after attachment of AMD the zeta potential for all liposomes tested rose as the AMD is positive. However, when dialysis was performed to remove excess AMD not adhered to the liposomes, only the ATBL liposomes retained their increased zeta potential (FIG. 5). For both liposomes with electrostatic adherence, the connection of the AMD to the liposomes was too weak to withstand dialysis and the positive charge (that is the AMD itself) was lost. The retention of AMD only on liposomes with covalent conjugation was confirmed by Thin Layer Chromatography (TLC) of the liposome both before and after dialysis. If electrostatic adsorbance was insufficient to withstand the minimal stress of dialysis, it certainly would not withstand a trip through the bloodstream. This analysis also concluded that at minimum 0.1 mg/ml of AMD were adhered to the nanoparticles.

### Example 4: ATBL Homing to the Bone Marrow Through CXCR4

Biodistribution of the AMD targeted nanoparticle was further tested. Non-targeted liposomes and AMD-liposomes were loaded with gadolinium (Gd) and administered (0.1×10\hat{ }13 particles) to BALBC mice intravenously. Gd accumulation in the bone was monitored by ICP (Inductively Coupled Plasma) Spectroscopy. The Gd concentration in the bone was higher with AMD targeting (FIG. 6A).

Next, non-targeted liposomes, liposomes with AMD electrostatically adsorbed to the surface and liposomes with AMD covalently linked to the surface were tagged with rhodamine. The different types of liposomes were intravenously injected into SCID mice bearing RPMI 8226 multiple myeloma cancer cells, and bone marrow was analyzed 24 hours later by flow cytometry. Significantly increased accumulation of rhodamine was observed in both bone marrow cells (FIG. 6B) and cancer cells (FIG. 6C) when the AMD-liposomes with covalent conjugation were administered. There was no difference in rhodamine accumulation between the untargeted liposomes and those with electrostatic adherence of AMD; again indicating that electrostatic attachment of AMD is insufficient to produce CXCR4 targeting in vivo.

Multiple myeloma is not the only disease of the bone marrow for which the AMD covalently conjugated liposomes are therapeutically useful. Acute myeloid leukemia and its subtype acute promyelocytic leukemia (APML) are characterized by an accumulation of immature white blood cells (promyelocytes) in the bone marrow. The HL60 cell line is a promyelocytic leukemia cell line that can be used as model for AML/APML. HL60 cells are positive for CXCR4 but at less than half the levels observed in MM cells (FIG. 7A). Rhodamine tagged liposomes were IV injected to mice bearing HL60 cells as was performed for the MM cells and after 24 hours accumulation of the liposomes in the bone marrow and cancer cells in the bone marrow was quantified. Even with the lower expression level of CXCR4 in the AML cells, the AMD liposomes accumulated to a significantly greater extent in both bone marrow cells (FIG. 7B) and AML cells in the bone marrow (FIG. 7C) than did the non-targeted liposomes. This demonstrates the utility of liposomes with covalently conjugated AMD for treating diseases of the bone marrow in general and not just multiple myeloma.

The AMD-liposomes of the invention are also therapeutically effective against bone metastasis. To test this cancer cells known to metastasize to the bone, e.g., 4T-Bone cells, are implanted to the mammary fat pad. After 16 days the primary tumor is removed and after 40 days bone metastasis appears. Once metastasis appears, AMD-liposomes loaded with an anticancer drug (e.g., a chemotherapeutic such as doxorubicin) are administered. Non-targeted liposomes with the same drug, the free drug, and empty AMD-liposomes are used as controls for determining the efficacy of the loaded AMD-liposomes. As with the other cancers tested, loaded AMD-liposomes inhibit tumor growth and are superior to both free drug and loaded untargeted liposomes. Biodistribution assays using rhodamine tagged liposomes are also performed to confirm accumulation of the AMD-liposomes in the 4T-Bone cells in the bone marrow.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A lipid nanoparticle comprising an outer surface covalently conjugated to 1,4-Bis[(1,4,8,11-tetraazacyclotetradecan-1-yl)methyl]benzene (AMD3100) or a derivative thereof capable of binding to C-X-C chemokine receptor type 4 (CXCR4), wherein said lipid nanoparticle comprises PEG and said covalently conjugated is via an amide bond to a carboxyl group on said PEG.

2. (canceled)

3. The nanoparticle of claim 1, wherein said lipid nanoparticle is a liposome.

4. The nanoparticle of claim 1, wherein said lipid nanoparticle comprises a lipid, cholesterol, and polyethylene glycol (PEG)-lipid.

5. The nanoparticle of claim 1, wherein said lipid nanoparticle comprises hydrogenated soybean phosphatidylcholine (HSPC), cholesterol, and 1,2-Distearoyl-sn-glycero-3-phosphorylethanolamine (DSPE) covalently bound to PEG.

6. The nanoparticle of claim 5, comprising DSPE-PEG1000 and wherein said HSPC, cholesterol, DSPE-PEG 1000 are present at a molar ratio of 55:40:5, respectively.

7. (canceled)

8. The nanoparticle of claim 71, where conjugation is via EDC carbodiimide crosslinking with S-NHS between a carboxyl group on said PEG and an amide in said AMD3100.

9. (canceled)

10. The nanoparticle of claim 1, comprising an aqueous interior comprising said a drug.

11. (canceled)

12. The nanoparticle of claim 1, wherein further comprising an anticancer drug.

13. The nanoparticle of claim 12, wherein said anticancer drug is a proteasome inhibitor.

14. The nanoparticle of claim 13, wherein said proteasome inhibitor is bortezomib.

15. The nanoparticle of claim 10, wherein said aqueous interior comprises a basic pH, optionally wherein said basic pH is about 9.5.

16. The nanoparticle of claim 14, comprising a bortezomib concentration of about 1 mg/ml.

17. (canceled)

18. (canceled)

19. A pharmaceutical composition comprising a nanoparticle of claim 1 and a pharmaceutically acceptable carrier, excipient or adjuvant.

20. A method of treating a CXCR4 positive cancer in a subject in need thereof, the method comprising, administering to said subject therapeutically effective dose of the nanoparticle of claim 12, thereby treating a CXCR4 positive cancer.

21. The method of claim 20, wherein said CXCR4 positive cancer is selected from multiple myeloma and acute myeloid leukemia.

22. (canceled)

23. The method of claim 20, wherein said nanoparticle comprises bortezomib and said dose is equivalent to a mouse dose of between 1-3 mg/kg body weight.

24. (canceled)

25. (canceled)

26. A method of determining suitability of a subject to be treated by a method of claim 20, the method comprising receiving a sample from said subject comprising cancer cells and measuring expression of CXCR4 on a surface of said cancer cells, wherein expression of CXCR4 above a predetermined threshold indicates the subject is suitable to be treated by a method of claim 20.

27. A method of covalently linking a molecule comprising a secondary amine to a lipid nanoparticle, the method comprising:
a. providing a lipid nanoparticle comprising at least one carboxyl group;
b. reacting said provided lipid nanoparticle with sulfo-N-hydroxysulfosuccinimide (S-NHS) and a carbodiimide coupling agent to obtain S-NHS active ester; and
c. reacting said S-NHS active ester with a molecule comprising a secondary amine;
thereby covalently linking said secondary amine to the lipid nanoparticle.

28. The method of claim 27, wherein at least one of:
a. said covalently linking comprises forming an amide bond between said secondary amine and the carboxy of the lipid nanoparticle;
b. said lipid nanoparticle comprises PEG comprising said at least one carboxyl group, and the method comprises producing an amide bond between said PEG and said molecule; and
c. said secondary amine is a tertiary amide upon linking.

29. (canceled)

30. (canceled)

31. A method of targeting an agent to bone marrow in a subject, the method comprising loading said agent into a nanoparticle of claim 1 to produce a loaded nanoparticle and administering said loaded nanoparticle to said subject, thereby targeting an agent to bone marrow in a subject, optionally wherein said administering is systemic administration, optionally wherein said systemic administration is intravenous administration.

32. (canceled)
